(19) United States
(12) Patent Application Publication
(10) Pub. No.: US 2019/0254941 A1
(43) Pub. Date: Aug. 22, 2019

(54) DOUBLE EMULSIONS COMPRISING A GELLED FATTY PHASE

(71) Applicant: CAPSUM, Marseille (FR)

(72) Inventors: Mathieu Goutayer, Saint Malo (FR); Yan Eric Pafumi, Gardanne (FR); Amélie Pujol, Marseille (FR)

(21) Appl. No.: 16/344,371

(22) PCT Filed: Oct. 25, 2017
(86) PCT No.: PCT/EP2017/077356

§ 371 Date: Apr. 24, 2019

(30) Foreign Application Priority Data
Oct. 26, 2016 (FR) 16 60386

(51) Int. Cl. A61K 8/06; A61K 8/58

(52) U.S. Cl. CPC A61K 8/066; A61K 8/585

(57) ABSTRACT

The present invention concerns a water-in-oil-in-water emulsion comprising an external continuous aqueous phase and a water-in-oil emulsion in the form of droplets (G1), each droplet (G1) comprising a continuous fatty phase and at least one droplet (G2) comprising an internal aqueous phase, said continuous fatty phase containing at least one gelling agent, said droplets (G1) comprising a shell formed of at least one anionic polymer (PA1) and at least one cationic polymer (PC).

The subject of the present invention concerns double water-in-oil-in-water emulsions having a gelled fatty phase and in which the internal fatty and aqueous phases are in droplet form. A further subject is a method to prepare the same and the use thereof in cosmetic compositions.

At the current time, the encapsulation of hydrophilic compounds is on the increase. Encapsulation technologies concern a broad variety of industrial sectors such as medicine, pharmaceuticals, food or cosmetics. Encapsulating a hydrophilic compound such as a cosmetic active substance entails insulating the latter from the outer medium. This strategy is especially required when this compound is incompatible with the other elements of the aqueous phase. Several methods have been developed to best adapt to different applications, such as spray-drying or coacervation.

However, there is a constant strive for the development of novel systems allowing the satisfactory encapsulation of hydrophilic compounds.

It is the objective of the present invention to provide a double, particularly macroscopic, emulsion allowing the encapsulation of hydrophilic compounds.

More particularly, it is the objective of the present invention to provide a double, particularly macroscopic, emulsion having satisfactory encapsulating properties of hydrophilic compounds combined with unique visual appeal and/or texture that are particularly attractive for consumers.

The present invention therefore concerns a water-in-oil-in-water emulsion comprising an external continuous aqueous phase and, as dispersed phase, a water-in-oil-in-water emulsion in the form of droplets (G1), each droplet (G1) comprising a continuous fatty phase and at least one, and preferably a single droplet (G2) comprising an internal aqueous phase, said continuous fatty phase containing at least one gelling agent.

said droplets (G1) having a shell formed of at least one anionic polymer (PA1) and at least one cationic polymer (PC).

Preferably, the above-mentioned droplets (G2) comprise a shell formed of at least one anionic polymer (PA2), the same as or differing from (PA1), and of at least one cationic polymer (PC).

The invention therefore concerns double water-in-oil-in-water emulsions having a gelled fatty phase. Preferably, in one emulsion of the invention, the droplets (G1) and (G2) are macroscopic i.e. visible to the naked eye.

In the present invention, the above-mentioned emulsions can also be designated by the term “dispersions”.

The emulsions of the invention comprise:
an internal aqueous phase,
an intermediate gelled fatty phase, and
an external aqueous phase.

These emulsions, in the external aqueous phase, comprise droplets (G1) of gelled fatty phase having a shell formed by coacervation, each droplet (G1) of gelled fatty phase containing at least one droplet (G2) of internal aqueous phase, this droplet (G2) of internal aqueous phase optionally comprising a shell formed by coacervation.

An emulsion of the invention is of particular advantage first in that it ensures particularly satisfactory encapsulation of hydrophilic compounds by means of droplets (G2), but also of lipophilic compounds by means of droplets (G1), and secondly has unique visual appeal and texture in the field of double emulsions which, for obvious reasons, meets continuing consumer demand in this respect.

An emulsion of the invention is also of particular interest with regard to kinetic stability, since the droplets (G1) or (G2) remain intact over a timescale of longer than one week, even longer than 1 month and even longer than 3 months, at ambient temperate e.g. T=25° C.±2° C., and at ambient pressure e.g. 1013 mbar.

In this respect, it was not obvious that double emulsions could be stable at ambient temperature e.g. T=25° C.±2° C., and at ambient pressure e.g. 1 013 mbar.

This advantageous property in terms of kinetic stability is all the more unexpected since the shell of droplets (G1), even of droplets (G2), described in detail below, is very thin. Therefore, no resistance related to rupture of the shell is felt by the user when applying onto keratinous material and no residual deposit of said shell is ascertained. The term evanescent shell can therefore be used.

The droplets (G1), even droplets (G2), given the type and properties of their shells, therefore differ from solid capsules i.e. capsules having a solid membrane such as those described for example in WO 2010/063937.

Additionally, the microfluidic method used to produce an emulsion of the invention allows the formation of macroscopic droplets (G1) and (G2) that are monodisperse. Also, the microfluidic method provides full control over the contents of each phase formed and hence over the concentrations of the encapsulated active substances.

According to the invention, the pH of an emulsion is typically between 4.0 and 8.0, in particular between 5.0 and 7.0.

The invention further concerns the use of an emulsion of the invention to prepare a composition, in particular a cosmetic composition. An emulsion of the invention, even a composition containing the same, can also be dedicated to the fields of medicine, pharmaceuticals or (agri)-foods.

The invention therefore also concerns a composition, particularly cosmetic, comprising at least one emulsion of the invention and in particular a physiologically acceptable medium.

Viscosity

The viscosity of the emulsions of the invention can vary widely allowing diverse textures to be obtained.

In one embodiment, an emulsion of the invention has viscosity of 1 mPa·s to 500 000 mPa·s, preferably 10 mPa·s to 300 000 mPa·s, better still 400 mPa·s to 100 000 mPa·s, and more particularly 1 000 mPa·s to 30 000 mPa·s, such as measured at 25° C.

Viscosity is measured at ambient temperature e.g. T=25° C.±2° C., and at ambient pressure e.g. 1 013 mbar, with the following method.

A viscometer of Brookfield type is used, typically a digital Brookfield RVDV-E viscometer (spring torque of 7187.0 dyne-cm); it is a rotating viscometer of set speed equipped with a spindle. A set speed is applied to the rotating spindle and measurement of the torque exerted on the spindle allows determination of viscosity with knowledge of the geometry/shape parameters of the spindle used.

For example, a spindle of size No. 04 is used (Brookfield reference: RV4). The shear rate corresponding to viscosity measurement is defined by the spindle used and the rotation speed thereof.

Viscosity measurement is carried out over 1 minute at ambient temperature (T=25° C.±2° C.). About 150 g of solution are placed in a beaker of 250 mL volume capacity having a diameter of about 7 cm so that the height of the volume occupied by the 150 g of solution is sufficient to arrive at the gauge mark on the spindle. The viscometer is set in operation at a speed of 10 rpm and it is waited until the value displayed on the screen becomes stable. This measurement gives the viscosity of the tested fluid, such as mentioned in the present invention.

External Continuous Aqueous Phase

As previously indicated, the emulsions of the invention comprise an external continuous aqueous phase, preferably in gel form, in particular a gel having viscosity adapted to suspend the droplets (G1) and thereby contribute towards the unique visual appeal of an emulsion of the invention.

In one embodiment, this aqueous phase has viscosity of between 1 mPa·s and 500 000 mPa·s, preferably between 10 mPa·s and 300 000 mPa·s, better still between 400 mPa·s and 100 000 mPa·s, and more particularly between 1 000 mPa·s and 30 000 mPa·s, such as measured at 25° C.

This viscosity is measured with the above-described method.

The external continuous aqueous phase of the emulsions at least comprises water.

Aside from distilled or deionized water, one water suitable for the invention can also be naturally sourced or floral water.

In one embodiment, the weight percent of water in the external continuous aqueous phase is at least 30%, preferably at least 40%, more preferably at least 50%, and further preferably it is at least 60%, in particular between 70% and 98%, and preferably between 55% and 95%, more preferably between 75% and 85%, relative to the total weight of said external aqueous phase.

The external continuous aqueous phase of the emulsion of the invention may also comprise at least one base. It may comprise a single base or a mixture of several different bases. If the external continuous aqueous phase of an emulsion of the invention comprises at least one pH-sensitive gelling agent, the presence of at least one base in said continuous aqueous phase particularly contributes towards enhancing the viscosity thereof.

In one embodiment, the base contained in the aqueous phase is a mineral base.

In one embodiment, the mineral base is selected from the group formed by hydroxides of alkali metals and hydroxides of alkaline-earth metals.

Preferably, the mineral base is a hydroxide of alkali metals and particularly NaOH.

In another embodiment, the base contained in the external aqueous phase is an organic base. Among organic bases, mention can be made of ammonia for example, pyridine, triethanolamine, aminomethyl propanol or triethylamine.

An emulsion of the invention may comprise from 0.01 to 10 weight %, preferably from 0.01 to 5 weight %, more preferably from 0.02 to 1 weight % of base, preferably of mineral base and particularly NaOH, relative to the total weight of said emulsion.

In one embodiment, the emulsions of the invention, even the compositions containing the same, do not comprise a surfactant.

Droplets (G1)

As indicated above, the double emulsions of the invention, as dispersed phase, comprise a water-in-oil emulsion in the form of droplets (G1).

A droplet (G1) of the invention is composed of a core, also called the inside of the droplet, surrounded by a shell which insulates the inside of the droplet from the external aqueous phase of the emulsion.

In one embodiment, the size of the droplets (G1) is greater than 500 μm, even greater than 1 000 μm, and better still between 500 μm and 3 000 μm, preferably between 1 000 μm and 2 000 μm, in particular between 800 μm and 1 500 μm.

In the present invention, the term “size” designates the diameter, in particular the mean diameter of the droplets.

In one embodiment, an emulsion of the invention is obtained with a microfluidic method such as defined below. As a result, the droplets (G1) have uniform size distribution. Preferably, the fatty phase of the emulsions of the invention is composed of a population of monodisperse droplets (G1), particularly such that they have a mean diameter D̅ of between 500 μm and 3000 μm and a coefficient of variation Cv of less than 10%, even less than 3%.

In the present invention, by “monodisperse droplets” is meant the fact that the population of droplets in the dispersed phase of the invention has uniform size distribution. Monodisperse droplets exhibit good monodispersity. Conversely, droplets having poor monodispersity are called “polydisperse”.

In one embodiment, the mean diameter D of the droplets is measured for example by analyzing a photograph of a batch of N droplets, using image processing software (Image J). Typically, according to this method, the diameter is measured in pixels, then expressed in μm in relation to the size of the container containing the droplets of the dispersion.

Preferably, the value of N is chosen to be equal to or higher than 30, so that this analysis reflects the distribution of the diameters of the droplets in said emulsion, in statistically significant manner.

The diameter Di of each droplet is measured, and the mean diameter D̅ is obtained by calculating the arithmetic mean of these values:

$$\overline{D} = \frac{1}{N} {\sum }_{i = 1}^{N} {D}_{i}$$

From these values Dᵢ, it is also possible to obtain the standard deviation σ of the droplet diameters in the dispersion:

$$σ = \sqrt{\frac{{\sum }_{i = 1}^{N} {( {D}_{i} - \overline{D} )}^{2}}{N}}$$

The standard deviation 6 of a dispersion reflects the distribution of the diameters Dᵢ of the droplets in the dispersion around the mean diameter D̅.

Knowing the mean diameter D̅ and standard deviation 6 of a dispersion, it is possible to determine that 95.4% of the droplet population is found within the range of diameters [D̅−2σ; D̅+2σ] and that 68.2% of the population is found in the range [D̅−σ; D̅+σ]

To characterize the monodispersity of the dispersion according to this embodiment, the coefficient of variation can be calculated:

$${C}_{v} = \frac{σ}{\overline{D}}$$

This parameter reflects the distribution of the diameters of the droplets as a function of the mean diameter thereof.

The coefficient of variation Cv of the diameters of the droplets (G1) according to this embodiment of the invention is lower than 10%, preferably lower than 5%, even lower than 3%.

In particular, the emulsions of the invention comprise from 0.1% to 70%, preferably from 0.5% to 65%, in particular from 1% to 60%, better still from 3% to 50%, and more particularly from 5% to 20% by weight of droplets (G1) (i.e. formed of the continuous fatty phase and internal aqueous phase) relative to the total weight of said emulsion.

As indicated above, each droplet (G1) comprises a fatty phase corresponding to the fatty phase of the emulsions of the invention.

Fatty Phase

As previously indicated, the fatty phase of the droplets (G1) comprises at least one gelling agent. Said gelling agent differs from the anionic polymers, cationic polymers, texturing agents, oils and additional compounds described below.

Gelling Agent

The presence of at least one gelling agent in the fatty phase of droplets (G1) contributes towards (i) suspending droplet(s) (G2) within each droplet (G1) and (ii) reinforcing the kinetic stability of an emulsion of the invention, and in particular the stability of tdroplets (G1) and (G2). Therefore, the droplets (G1) and (G2) are able to remain intact over a timescale longer than 1 month, in particular longer than 3 months, even longer than 6 months, at ambient temperature e.g. T=25° C.±2° C., and at ambient pressure e.g. 1 013 mbar.

In the invention and unless otherwise indicated, by «gelling agent» is meant an agent allowing increased viscosity of the fatty phase of droplets (G1) compared with an emulsion devoid of said gelling agent, allowing a final viscosity of the gelled fatty phase to be reached that is higher than 20 000 mPa·s, preferably higher than 50 000 mPa·s, more preferably higher than 100 000 mPa·s, and further preferably higher than 200 000 mPa·s.

Preferably, the viscosity of the fatty phase of droplets (G1) of the emulsion in the presence of said gelling agent is between 20 000 and 100 000 000 mPa·s, more preferably between 50 000 and 1 000 000 mPa·s, and further preferably between 100 000 and 500 000 mPa·s, at 25° C.

The choice of gelling agent(s) is made having particular regard to the type of fatty phase of the droplets (G1) and/or desired results, notably in terms of sensory aspect and/or texture. For obvious reasons of compatibility, the gelling agent is lipophilic or liposoluble.

In one particular embodiment, when the fatty phase of droplets (G1) also contains at least one oil, in particular such as described below, the choice of oil(s) is made having regard to the type of gelling agent(s), and conversely. The oil(s) used must be a satisfactory solvent of the gelling agent. This choice is within the reach of persons skilled in the art.

In one embodiment, the emulsions of the invention comprise from 0.5% to 99.99%, preferably from 1% to 70%, in particular from 1.5% to 50%, better still from 2% to 40%, in particular from 2.5% to 30%, and further preferably from 10% to 20% by weight of gelling agent(s) relative to the total weight of the fatty phase of droplets (G1).

In one embodiment, the gelling agent is selected from among organic or mineral, polymeric or molecular lipophilic gelling agents, fats solid at ambient temperature and pressure, and mixtures thereof.

Lipophilic Gelling Agent(s)

The gelling agents which can be used in the invention can be organic or mineral, polymeric or molecular lipophilic gelling agents.

In one embodiment, the gelling agent is selected from the group formed by modified clays, silicas such as pyrogenated silica and mixtures thereof.

As mineral lipophilic gelling agent, mention can be made of optionally modified clays such as hectorites modified with a C₁₀ to C₂₂ ammonium chloride such as hectorite modified with distearyl dimethyl ammonium chloride e.g. the one marketed under the trade name Bentone 38V® by ELEMENTIS. Mention can also be made of hectorite modified with distearyl dimethyl ammonium chloride also known as quaternium-18 bentonite e.g. the products marketed or produced under the trade names Bentone 34 by Rheox, Claytone XL, Claytone 34 and Claytone 40 marketed or produced by Southern Clay, modified clays known under the names of benzalkonium and quaternium-18 bentonites and marketed or produced under the trade names Claytone HT, Claytone GR and Claytone PS by Southern Clay, clays modified with stearyl dimethyl benzoyl ammonium chloride known as stearalkonium bentonites e.g. the products marketed or produced under the trade names Claytone APA and Claytone AF by Southern Clay, and Baragel 24 marketed or produced by Rheox.

Pyrogenated silica can also be cited, optionally with hydrophobic surface treatment having a particle size smaller than 1 μm. It is possible chemically to modify the surface of silica via chemical reaction generating a reduction in the number of silanol groups on the silica surface. In particular, silanol groups can be substituted by hydrophobic groups: a hydrophobic silica is thereby obtained.

The hydrophobic groups can be:
trimethylsiloxyl groups, notably obtained by treating pyrogenated silica in the presence of hexamethyldisilazane. Silicas thus treated are called «Silica silylate» in the CTFA dictionary (8th Edition, 2000). For example, they are marketed under the references Aerosil R812® by DEGUSSA, CAB-O-SIL TS-530® by CABOT; or
dimethylsilyloxyl or polydimethylsiloxane groups, obtained in particular by treating pyrogenated silica in the presence of polydimethylsiloxane or dimethyldichlorosilane. Silicas thus treated are called «Silica dimethyl silylate» in the CTFA dictionary (8^{the} Edition, 2000). For example, they are marketed under the references Aerosil R972® and Aerosil R974® by DEGUSSA, CAB-O-SIL TS-610® and CAB-O-SIL TS-720® by CABOT.

Hydrophobic pyrogenated silica particularly has a particle size that can be nanometric or micrometric e.g. ranging from about 5 to 200 nm.

Organic, polymeric lipophilic gelling agents are for example partly or fully crosslinked elastomeric organopolysiloxanes of three-dimensional structure such as those marketed under the trade names KSG6®, KSG16® and KSG18® by SHIN-ETSU, under the names Dow Corning® EL-7040, Trefil E-505C® and Trefil E-506C® by DOW-CORNING, the names Gransil SR-CYC®, SR DMF10®, SR-DC556®, SR SCYC gel®, SR DMF 10 gel® and SR DC 556 gel® by GRANT INDUSTRIES, the names SF 1204® and JK 113® by GENERAL ELECTRIC; ethyl cellulose such as the one sold under the trade name Ethocel® by DOW CHEMICAL; galactomannans having from one to six and in particular two to four hydroxyl groups per ose, substituted by a saturated or unsaturated alkyl chain, such as guar gum alkylated with C₁ to C₆ alkyl chains, particularly C₁ to C₃, and mixtures thereof. Block copolymers of «diblock», «triblock» or «radial» type e.g. of polystyrene/polyisoprene, polystyrene/polybutadiene type marketed for example under the trade name Luvitol HSB® by BASF, of polystyrene/copoly(ethylene-propylene) type such as those marketed under the trade name Kraton® by SHELL CHEMICAL CO, or of polystyrene/copoly(ethylene-butylene) type, the mixtures of triblock and radial (star) copolymers in isododecane such as those marketed by PENRECO under the trade name Versagel® e.g. the mixture of butylene/ethylene/styrene triblock copolymer and ethylene/propylene/styrene star copolymer in isododecane (Versagel M 5960).

In one embodiment, the gelling agents which can be used in the invention can be selected from the group formed by polyacrylates; esters of sugar/polysaccharide and fatty acid(s), in particular esters of dextrin and fatty acid(s), esters of inulin and fatty acid(s), or esters of glycerol and fatty acid(s); polyamides; and mixtures thereof.

As lipophilic gelling agent, polymers of weight average molecular weight lower than 100 000 can also be cited, comprising a) a polymeric backbone having hydrocarbon repeating units provided with at least one heteroatom, and optionally b) at least one pendant fatty chain and/or at least one end fatty chain, optionally functionalized, having 6 to 120 carbon atoms and bonded to these hydrocarbon repeating units such as described in applications WO 02/056847, WO 02/47619, in particular polyamide resins (particularly comprising alkyl groups having 12 to 22 carbon atoms) such as those described in U.S. Pat. No. 5,783,657.

As an example of polyamide resin able to be used in the present invention, mention can be made of UNICLEAR 100 VG® marketed by ARIZONA CHEMICAL.

It is also possible to use silicone polyamides of polyorganosiloxane type such as those described in U.S. Pat. Nos. 5,874,069, 5,919,441, 6,051,216 and 5,981,680.

These silicone polymers can belong to the two following families:
polyorganosiloxanes comprising at least two groups capable of setting up hydrogen interactions, these two groups being located in the chain of the polymer; and/or
polyorganosiloxanes comprising at least two groups capable of setting up hydrogen interactions, these two groups being located on grafts or branches.

Among the lipophilic gelling agents able to be used in the present invention further mention can be made of the esters of dextrin and fatty acid, such as dextrin palmitates.

In one embodiment, the ester of dextrin and fatty acid(s) of the invention is a mono- or poly-ester of dextrin and at least one fatty acid meeting following formula (II):

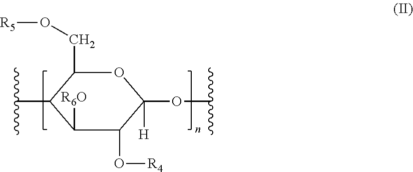

where:
n is an integer ranging from 2 to 200, preferably ranging from 20 to 150, and in particular ranging from 25 to 50,
the radicals R₄, R₅ and R₆, the same or different, are selected from among hydrogen or —CORₐ acyl group where the radical Rₐ is a straight-chain or branched, saturated or unsaturated hydrocarbon radical having 5 to 50, preferably 5 to 25 carbon atoms,
   provided that at least one of said radicals R₄, R₅ or R₆ differs from hydrogen.

In one embodiment, R₄, R₅ and R₆ are each independently H or —CORₐ acyl group where Rₐ is a hydrocarbon radical such as previously defined, provided that at least two of said radicals R₄, R₅ or R₆ are the same and differ from hydrogen.

In one embodiment, when the radicals R₄, R₅ and R₆, the same or different are a —CORₐ radical, these can be selected from among caprylyl, caproyl, lauroyl, myristyl, palmityl, stearyl, eicosanyl, docosanoyl, isovaleryl, ethyl-2 butyryl, ethylmethylacetyl, isoheptanyl, ethyl-2 hexanyl, isononanyl, isodecanyl, isotridecanyl, isomyristyl, isopalmityl, isostearyl, isohexanyl, decenyl, dodecenyl, tetradecenyl, myristyl, hexadecenoyl, palmitoleyl, oleyl, elaidyl, eicosenyl, sorbyl, linoleyl, linolenyl, punicyl, arachidonyl, stearolyl radicals, and mixtures thereof.

Among the esters of dextrin and fatty acid(s) mention can be made for example of dextrin palmitates, dextrin myristates, dextrin palmitates/ethylhexanoates and mixtures thereof.

Particular can be made of the esters of dextrin and fatty acid(s) marketed under the trade names Rheopearl® KL2 (INCI name: dextrin palmitate), Rheopearl® TT2 (INCI name: dextrin palmitate ethylhexanoate), and Rheopearl® MKL2 (INCI name: dextrin myristate) by Miyoshi Europe.

Among the lipophilic gelling agents able to be used in the present invention, further mention can be made of the esters of inulin and fatty acids.

Particular mention can be made of the esters of inulin and fatty acid(s) marketed under the trade names Rheopearl® ISK2 or Rheopearl® ISL2 (INCI name: Stearoyl Inulin) by Miyoshi Europe.

In one embodiment, the gelling agent is selected from among the polyacrylates resulting from the polymerization of C₁₀-C₃₀ alkyl acrylate(s), preferably C₁₄-C₂₄ alkyl acrylate(s) and more preferably C₁₈-C₂₂ alkyl acrylate(s).

In one embodiment, the polyacrylates are polymers of acrylic acid esterified with a fatty alcohol comprising a saturated carbon chain having 10 to 30 carbon atoms preferably 14 to 24 carbon atoms, or a mixture of said fatty alcohols. Preferably the fatty alcohol has 18 carbon atoms or 22 carbon atoms.

Among the polyacrylates, particular mention can be made of stearyl polyacrylate, behenyl polyacrylate. Preferably the gelling agent is stearyl polyacrylate or behenyl polyacrylate.

The polyacrylates marketed under the trade names Intelimer® (INCI name: Poly C₁₀-C₃₀ alkyl acrylate) can be cited, in particular Intelimer® 13.1 and Intelimer® 13.6 by Airproducts.

In one embodiment, the gelling agent is an ester of glycerol and fatty acid(s), in particular a mono-, di- or tri-ester of glycerol and fatty acid(s). Typically, said ester of glycerol and fatty acid(s) can be used alone or in a mixture.

In the invention it may be an ester of glycerol and a fatty acid or an ester of glycerol and a mixture of fatty acids.

In one embodiment, the fatty acid is selected from the group formed by behenic acid, isooctadecanoic acid, stearic acid, eicosanoic acid, and mixtures thereof.

In one embodiment, the ester of glycerol and fatty acid(s) has the following formula (III):

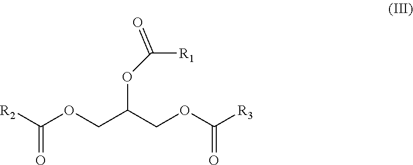

where: R₁, R₂ and R₃ are each independently selected from among H and a saturated alkyl chain having 4 to 30 carbon atoms, at least one of R₁, R₂ and R₃ differing from H.

In one embodiment, R₁, R₂ and R₃ are different.

In one embodiment, R₁, R₂ and/or R₃ are a saturated alkyl chain having 4 to 30, preferably 12 to 22, more preferably 18 to 22 carbon atoms.

In one embodiment the ester of glycerol and fatty acid(s) corresponds to a compound of formula (III) where R₁═H, R₂═C₂₁H₄₃ et R₃═C₁₉H₄₀.

In one embodiment, the ester of glycerol and fatty acid(s) corresponds to a compound of formula (III) where R₁═R₂═R₃═C₂₁H₄₃.

In one embodiment, the ester of glycerol and fatty acid(s) corresponds to a compound of formula (III) where R₁═R₂═H, and R₃═C₁₉H₄₀.

In one embodiment, the ester of glycerol and fatty acid(s) corresponds to a compound of formula of (III) where R₁═R₂═H, and R₃═C₁₇H₃₅.

Particular mention can be made of the esters of glycerol and fatty acid(s) marketed under the trade names Nomcort HK-G (INCI name: Glyceryl behenate/eicosadioate) and Nomcort SG (INCI name: Glyceryl tribehenate, isostearate, eicosadioate), by Nisshin Oillio.

Wax(es)

By «wax» in the meaning of the invention is meant a lipophilic compound solid at ambient temperature (25° C.), having reversible solid state/liquid state change and a melting point of 30° C. or higher possibly reaching 120° C.

The protocol for measuring this melting point is described below.

The waxes able to be used in an emulsion of the invention can be selected from among solid waxes, deformable or not at ambient temperature, of animal, vegetable or mineral origin, or synthesized, and mixtures thereof.

In particular, hydrocarbon waxes can be used such as beeswax, lanolin wax, Chinese insect waxes; rice wax, carnauba wax, Candelilla wax, Ouricury wax, esparto wax, cork fibre wax, sugarcane wax; Japan wax, sumac wax; montan wax, microcrystalline waxes, paraffins and ozokerite; polyethylene waxes, waxes obtained via Fisher-Tropsch synthesis and waxy copolymers and esters thereof.

Particular mention can be made of the waxes marketed under the trade names Kahlwax®2039 (INCI name: Candelilla cera) and Kahlwax®6607 (INCI name: Helianthus Annuus Seed Wax) by Kahl Wachsraffinerie, Casid HSA (INCI name: Hydroxystearic Acid) by SACI CFPA, Performa®260 (INCI name: Synthetic wax) and Performa®103 (INCI name: Synthetic wax) by New Phase, and AJK-CE2046 (INCI name: Cetearyl alcohol, dibutyl lauroyl glutamide, dibutyl ethylhaxanoyl glutamide) by Kokyu Alcohol Kogyo.

The waxes can also be cited obtained by catalytic hydrogenation of animal or vegetable oils having C₈-C₃₂ straight-chain or branched fatty chains.

Among these, particular mention is made of hydrogenated jojoba oil hydrogenated sunflower seed oil, hydrogenated castor oil, hydrogenated copra oil and hydrogenated lanolin oil, di-(trimethylol-1,1,1 propane) tetrastearate sold under the trade name «HEST 2T-4S» by HETERENE, di-(trimethylol-1,1,1 propane) tetrabehenate sold under the trade name HEST 2T-4B by HETERENE.

Use can also be made of the waxes obtained by transesterification and hydrogenation of vegetable oils such as castor or olive oil, e.g. the oils sold under the trade names Phytowax Castor 16L64® and 22L73® and Phytowax Olive 18L57 by SOPHIM. Said waxes are described in application FR 2 792 190.

Silicone waxes can also be used which, advantageously, can be substituted polysiloxanes, preferably with low melting point.

Among commercial silicone waxes of this type, particular mention is made of those sold under the trade names Abilwax 9800, 9801 or 9810 (GOLDSCHMIDT), KF910 and KF7002 (SHIN ETSU), or 176-1118-3 and 176-11481 (GENERAL ELECTRIC).

The silicone waxes able to be used may also be alkyl or alkoxydimethicones such as the following commercial products: Abilwax 2428, 2434 and 2440 (GOLDSCHMIDT), or VP 1622 and VP 1621 (WACKER), and (C₂₀-C₆₀) alkyldimethicones, in particular (C₃₀-C₄₅) alkyldimethicones such as the silicone wax sold under the trade name SF-1642 by GE-Bayer Silicones.

It is also possible to use hydrocarbon waxes modified by silicone or fluorinated groups such as: siliconyl candelilla, siliconyl beeswax and Fluorobeeswax by Koster Keunen.

The waxes can also be selected from among fluorinated waxes.

Butter(s) or Pasty Fats

By «butter» (also called «pasty fat») in the meaning of the invention is meant a lipophilic fatty compound with reversible solid/liquid state change and having a liquid fraction and a solid fraction at a temperature of 25° C. and at atmospheric pressure (760 mm Hg). In other words, the start melting point of the pasty compound may be lower than 25° C. The liquid fraction of the pasty compound measured at 25° C. can represent 9 to 97 weight % of the compound. This liquid fraction at 25° C. preferably represents between 15 and 85%, more preferably between 40 and 85% by weight. Preferably the butter(s) have an end-melt temperature lower than 60° C. Preferably the butter(s) have hardness equal to or lower than 6 MPa.

Preferably, the butters or pasty fats in the solid state have anisotropic crystalline organization seen under X-ray observation.

In the meaning of the invention, the melting point corresponds to the temperature of the most endothermic peak seen under differential scanning calorimetry (DSC) as described in standard ISO 11357-3; 1999. The melting point of a paste or wax can be measured using differential scanning calorimetry (DSC), for example using the calorimeter sold under the trade name “DSC 02000” by TA Instruments.

Regarding measurement of melting point and determination of end-of-melt temperature, the protocols for preparing samples and measurement are the following: a 5 mg sample of pasty fat (or butter) or wax, previously heated to 80° C. and taken up under magnetic agitation using a spatula, also heated, is placed in a hermetically sealed aluminium capsule or crucible. Two tests are conducted to ensure reproducibility of results.

Measurements are taken on the above-mentioned calorimeter. The oven is flushed with nitrogen. Cooling is ensured via the RCS 90 heat exchanger. The sample is then subjected to the following protocol after first being placed at a temperature of 20° C., then subjected to a first temperature rise ranging from 20° C. to 80° C. at a heating rate of 5° C./minute, then cooled from 80° C. to −80° C. at a cooling rate of 5° C./minute, and finally subjected to a second temperature rise ranging from −80° C. to 80° C. at a heating rate of 5° C./minute. During the second temperature rise, the variation is measured in the difference of energy absorbed by the empty crucible and the crucible containing the butter sample, as a function of temperature. The melting point of the compound is the value of the temperature corresponding to the apex of the peak in the curve representing the variation in difference of absorbed energy as a function of temperature. The end-of-melt temperature corresponds to the temperature at which 95% of the sample has melted.

The liquid fraction in weight of the butter (or pasty fat) at 25° C. is equal to the ratio between the enthalpy of fusion consumed at 25° C. and the enthalpy of fusion of the butter. The enthalpy of fusion of the butter or pasty compound is the enthalpy consumed by the compound to change from the solid state to the liquid state.

The butter is said to be in the solid state when the entirety of its mass is in solid crystalline form. The butter is said to be in the liquid state when the entirety of its mass is in liquid form. The enthalpy of fusion of the butter is equal to the integral of the entire fusion curve obtained using the above-mentioned calorimeter with a temperature rise of 5° C. or 10° C. per minute, as per standard ISO 11357-3:1999. The enthalpy of fusion of butter is the amount of energy needed to cause the compound to change from the solid state to the liquid state. It is expressed in J/g.

The enthalpy of fusion consumed at 25° C. is the amount of energy absorbed by the sample to change from the solid state to the state it exhibits at 25° C. formed of a liquid fraction and solid fraction. The liquid fraction of the butter measured at 32° C. preferably represents 30% to 100 weight % of the compound, preferably 50 to 100%, more preferably 60 to 100 weight % of the compound. When the liquid fraction of the butter measured at 32° C. is 100%, the temperature of the end of fusion range of the pasty compound is 32° C. or lower. The liquid fraction of the butter measured at 32° C. is equal to the ratio between the enthalpy of fusion consumed at 32° C. and the enthalpy of fusion of the butter. The enthalpy of fusion consumed at 32° C. is calculated in the same manner as the enthalpy of fusion consumed at 23° C.

Regarding measurement of hardness, the protocols for preparing samples and for measurement are the following: the emulsion of the invention or butter is placed in a mould 75 mm in diameter filled to about 75% of its height. To overcome thermal history and to control crystallization, the mould is placed in a programmable Vôtsch VC0018 oven where it is first placed at a temperature of 80° C. for 60 minutes, then cooled from 80° C. to 0° C. at a cooling rate of 5° C./minute, then left at the stabilized temperature of 0° C. for 60 minutes, subjected to a temperature rise ranging from 0° C. to 20° C. at a heating rate of 5° C./minute, and left at the stabilized temperature of 20° C. for 180 minutes. Measurement of the compression force is conducted with the TA/TX2i texture analyzer by Swantech. The probe used is selected according to texture: steel cylindrical probe 2 mm in diameter for very rigid raw materials; steel cylindrical probe 12 mm in diameter for scarcely rigid raw materials. Measurement comprises 3 steps: a 1^{st} step after automatic detection of the sample when the probe moves at a measuring speed of 0.1 mm/s, and enters the emulsion of the invention or the butter to a penetration depth of 0.3 mm, the software records the value of the maximum force reached; a 2^{nd} so-called relaxation step, when the probe remains at this position for one second and the force is recorded after 1 second of relaxation; finally a 3^{rd} so-called withdrawal step when the probe returns to its initial position at a rate of 1 mm/s and the withdrawal energy of the probe is recorded (negative force).

The hardness value measured at the first step corresponds to the maximum compressive force measured in Newtons divided by the surface area of the cylindrical probe of the texture analyzer expressed in mm² in contact with the butter or emulsion of the invention. The hardness value obtained is expressed in mega-pascals or MPa.

The pasty fat or butter can be selected from among synthetic compounds and compounds of vegetable origin, A pasty fat can be obtained by synthesis from starting products of vegetable origin.

The pasty fat is advantageously selected from among:
lanolin and derivatives thereof such as lanolin alcohol, oxyethylenated lanolins, acetylated lanolin, esters of lanolin such as isopropyl lanolate, oxypropylenated lanolins;
polymer or non-polymer silicone compounds such as polydimethysiloxanes of high molecular weight, polydimethysiloxanes with side chains of alkyl or alkoxy type having 8 to 24 carbon atoms, in particular stearyl dimethicones,
polymer or non-polymer fluorinated compounds;
vinyl polymers, in particular:
olefin homopolymers,
olefin copolymers,
homopolymers and copolymers of hydrogenated dienes,
straight-chain or branched oligomers, homo- or copolymers of alkyl (meth)acrylates preferably having a C₈-C₃₀ alkyl group,
homo oligomers and copolymers of vinyl esters having C₈-C₃₀ alkyl groups,
homo oligomers and copolymers of vinyl ethers having C₈-C₃₀ alkyl groups
liposoluble polyethers resulting from polyetherification between one or more C₂-C₁₀₀ diols, preferably C₂-C₅₀,
esters and polyesters, and
mixtures thereof.

In one preferred embodiment of the invention, the particular butter(s) of vegetable origin are such as those described in Ullmann's Encyclopaedia of Industrial Chemistry («Fats and Fatty Oils», A. Thomas, published on 15 Jun. 2000, D01: 10.1002/14356007.a10_173, item 13.2.2.2F. Shea Butter, Borneo Tallow, and Related Fats (Vegetable Butters)).

Particular mention can be made of C₁₀-C₁₈ triglycerides (INCI name: 010-18 Triglycerides) which, at a temperature of 25° C. and at atmospheric pressure (760 mm Hg), comprise a liquid fraction and a solid fraction: shea butter, Shea Nilotica butter (Butyrospermum parkii), Galam butter (Butyrospermum parkii), Borneo butter (or tengkawang tallow) (Shorea stenoptera), Shorea butter, illipe butter, Madhuca or Bassia Madhuca longifolia butter, mowrah butter (Madhuca latifolia), Katiau butter (Madhuca mottleyana), Phulwara butter (M. butyracea), mango butter (Mangifera indica), Murumuru butter (Astrocatyum murumuru), Kokum butter (Garcinia indica), Ucuuba butter (Virola sebifera), Tucuma butter, Painya butter (Kpangnan) (Pentadesma butyracea), coffee butter (Coffee arabica), apricot butter (Prunus armeniaca), Macadamia butter (Macadamia Temifolia), grapeseed butter (Vitis vinifera), avocado butter (Persea gratissima), olive butter (Olea europaea), sweet almond butter (Prunus amygdalus dulcis), cocoa butter (Theobroma cacao) and sunflower seed butter, butter under the INCI name Astrocaryum Murumuru Seed Butter, butter under the INCI name Theobroma grandiflorum Seed Butter, and the butter under the INCI name Irvingia gabonensis Kernel Butter, the esters of jojoba (mixture of jojoba wax and hydrogenated oil) (INCI name: Jojoba esters) and the ethyl esters of shea butter (INCI name: Shea butter ethyl esters), and mixtures thereof.

In one preferred embodiment, a gelling agent of the fatty phase of the droplets (G1) of the invention is a heat-sensitive gelling agent, namely which reacts to heat, and in particular it is a gelling agent solid at ambient temperature and liquid at a temperature higher than 40° C., preferably higher than 50° C.

Therefore, the gelling agent is preferably selected from among dextrin palmitates.

In another preferred embodiment, a gelling agent of the fatty phase of the droplets (G1) of the invention is a thixotropic gelling agent. This embodiment is advantageous in that an emulsion of the invention can be obtained by implementing a microfluidic method at ambient temperature. Therefore, the gelling agent is preferably selected from among pyrogenated silica optionally with surface hydrophobic treatment.

In one particular embodiment, an emulsion of the invention, in particular the fatty phase of the droplets (G1), does not comprise an elastomer gel comprising at least one dimethicone, particularly such as marketed by NuSil Technology under the trade name CareSil™ CXG-1104 (INCI: Dimethicone (and) Dimethicone/Vinyl Dimethicone Crosspolymer).

In one embodiment the fatty phase of the droplets (G1) may also comprise at least one oil. The fatty phase of the droplets (G1) in this embodiment can therefore be designated as an oily phase.

Oil(s)

The droplets (G1) of the invention may comprise a single oil, or a mixture of several oils. An emulsion of the invention may therefore comprise at least one, at least two, at least three, at least four, at least five even more oils such as described below.

By «oil» is meant a fat that is liquid at ambient temperature (25° C.).

As oils able to be used in the emulsion of the invention, mention can be made for example of:
hydrocarbon oils of vegetable origin, in particular such as described below;
hydrocarbon oils of animal origin such as perhydrosqualene and squalane;
synthetic esters and ethers, in particular of fatty acids, such as oils of formulas R₁COOR₂ and R₁OR₂ where R₁ is the remainder of a C₈ to 023 fatty acid, and R₂ is a C₃ to 030 hydrocarbon chain, whether or not branched, e.g. a Purcellin oil, isononyl isononanoate, isodecyl neopentanoate, isopropyl myristate, ethyl-2-hexyl palmitate, octyl-2-dodecyl stearate, octyl-2-dodecyl ercucate, isostearyl isostearate; hydroxylated esters such as isostearyl lactate, octylhydroxystearate, octyldodecyl hydroxystearate, diisostearyl-malate, triisocetyl citrate, heptanoates, octanoates, fatty alcohol decanoates; polyol esters such as propylene glycol dioctanoate, neopentylglycol diheptanoate and diethyleneglycol diisononanoate; and the esters of pentaerythritol such as pentaerythrityl tetrabehenate (DUB PTB) or pentaerythrityl tetraisostearate (Prisorine 3631);
straight-chain or branched hydrocarbons of mineral or synthetic origin such as paraffin oils whether or not volatile and derivatives thereof, vaseline, polydecenes, hydrogenated polyisobutene such as Parleam oil;
silicone oils e.g. polydimethylsiloxanes (PDMS) whether or not volatile with straight-chain or cyclic silicone chain, liquid or pasty at ambient temperature in particular cyclopolydimethylsiloxanes (cyclomethicones) such as cyclohexasiloxane and cyclopentasiloxane; polydimethylsiloxanes (or dimethicones) having alkyl, alkoxy or phenyl groups that are pendant or at the end of the silicone chain, groups having 2 to 24 carbon atoms; phenylated silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
fatty alcohols having 8 to 26 carbon atoms such as cetyl alcohol, stearyl alcohol and mixture thereof (cetylstearyl alcohol), or octyldodecanol;
fluorinated oils, part hydrocarbon and/or silicone, as described in JP-A-2-295912;
and mixtures thereof.

In one preferred embodiment, the oil is selected from among synthetic esters and ethers, preferably the esters of formula R₁COOR₂, where R₁ is the remainder of a C₈ to 023 fatty acid, and R₂ is a C₃ to C₃₀ hydrocarbon chain whether or not branched.

In one embodiment, the oil is selected from among fatty alcohols having 8 to 26 carbon atoms.

In one embodiment, the oil is selected from among hydrocarbon oils having 8 to 16 carbon atoms and in particular C₈-C₁₆ branched alkanes (also called isoparaff ins or isoalkanes), such as isododecane (also called 2-methylundecane), isodecane, isohexadecane, for example the oils sold under the trade names Isopars® or Permethyls®.

In one preferred embodiment, the oil is selected from the group formed by isononyl isononanoate, dimethicone, isohexadecane, polydimethylsiloxane, octyldodecanol, isodecyl neopentanoate and mixtures thereof.

In another preferred embodiment, the fatty phase of the droplets (G1) comprises an oil selected from among silicone oils. Preferably, the fatty phase of the droplets (G1) does not comprise other oils differing from silicone oils. Preferably the oils contained in the fatty phase of the droplets (G1) are silicone oils.

In one preferred embodiment, an emulsion of the invention comprises at least 1 weight % of oil(s) relative to the total weight of said emulsion.

In another embodiment, an emulsion of the invention, in particular the fatty phase of the droplets (G1), does not comprise polydimethylsiloxane (PDMS), and preferably does not comprise a silicone oil.

In another embodiment, an emulsion of the invention does not comprise a vegetable oil.

In a further embodiment of the invention, the fatty phase of the droplets (G1) of an emulsion of the invention comprises at least one hydrocarbon oil of vegetable origin. As vegetable oils, particular mention can be made of liquid triglycerides of 04-C₁₀ fatty acids, such as the triglycerides of heptanoic or octanoic acids, or for example sunflower seed, corn, soybean, pumpkin, grapeseed, sesame, hazelnut, apricot, macadamia arara, castor, and avocado oils, the triglycerides of caprylic/capric acids (INCI name: Caprylic/Capric Triglyceride) such as those marketed by Stearineries Dubois or those available under the trade names «Miglyol 810», «Miglyol 812» and «Miglyol 818» by Dynamit Nobel, jojoba oil, shea butter oil, and mixtures thereof.

Preferably, the vegetable oil is selected from among those high in polyunsaturated fatty acids. By “unsaturated fatty acid” in the meaning of the present invention is meant a fatty acid having at least one double bond. They are most particularly long-chain fatty acids i.e. having more than 14 carbon atoms. The unsaturated fatty acids can be in acid form or salt form e.g. their calcium salt, or in the form of derivatives in particular esters(s) of fatty acid(s).

Preferably, the vegetable oil is selected from among oils high in long-chain fatty acids i.e. having more than 14 carbon atoms, and better still unsaturated fatty acids having 18 to 22 carbon atoms, in particular w-3 and w-6 fatty acids. Therefore, advantageously, the vegetable oils are selected from among evening primrose, borage, blackcurrant seed, hemp, walnut, soybean, sunflower seed, wheatgerm, fenugreek, rose hip, echium, argan, baobab, rice bran, sesame, almond, hazelnut, chia, linseed, olive, avocado, safflower, coriander, rapeseed (particularly Brassica naptus) oils and mixtures thereof.

Preferably, the vegetable oil is selected from among matt non-shiny oils. In this respect, particular mention can be made of Moringa oil.

In a further embodiment, the fatty phase of the droplets (G1) of an emulsion of the invention comprises at least one non-volatile hydrocarbon oil (or H1 oil) containing more than 90%, preferably more than 95%, of fatty acids having a chain length of 18 carbon atoms or longer, preferably 20 carbon atoms or longer.

Preferably, more than 90%, and preferably more than 95% of the fatty acids of the non-volatile hydrocarbon oil have a chain length of between C₁₈ and C₃₈, preferably between C₂₀ and C₂₈, and better still between C₂₀ and 022.

By «non-volatile» is meant an oil having a vapour pressure at ambient temperature and atmospheric pressure that is nonzero and lower than 0.02 mm Hg (2.66 Pa) and better still lower than 10⁻³ mm Hg (0.13 Pa).

For example, as H1 oils, mention can be made of jojoba oil, linseed oil, Perilla oil, Inca Inchi oil, rose hip oil, rapeseed oil, hemp oil, sweet almond oil, corn oil, apricot oil, castor oil, Meadowfoam oil (INCI: Limnanthes Alba (Meadowfoam) Seed Oil) and mixtures thereof, preferably jojoba oil and/or Meadowfoam oil, and better still Meadowfoam oil.

The use of H1 oils, in particular of Meadowfoam oil, in the fatty phase of the droplets (G1) of an emulsion of the invention has advantageous effects in terms of reduced opacification of the continuous aqueous phase and/or reduced droplet adhesion to the walls of the packaging and/or reduced aggregation of the droplets together.

Advantageously, when the fatty phase of the droplets (G1) of an emulsion of the invention comprises at least one gelling agent selected from among the esters of sugar or polysaccharide and fatty acid(s), in particular of dextrin and fatty acid(s), and more particularly selected from the group formed by dextrin palmitates, dextrin myristates, dextrin palmitates/ethylhexanoates, and mixtures thereof, said fatty phase of the droplets (G1) also comprises at least one oil having a refractive index close to that of the gelling agent(s), namely an oil having a refractive index at ambient temperature (25° C.) and atmospheric pressure of between 1.2 and 1.8, preferably between 1.3 and 1.7, more preferably between 1.4 and 1.6 and further preferably between 1.45 and 1.55.

This embodiment is advantageous in that it allows improved transparency of the fatty phase of the droplets (G1), and hence transparency of the emulsion of the invention.

Advantageously, the oil having a refractive index of between 1.2 and 1.8 is a silicone oil, in particular a phenylated silicone oil.

As silicone oils conforming to the invention, mention can be made for example of polymethylsiloxanes (PDMS) whether or not volatile having a straight-chain or cyclic silicone chain, liquid or pasty at ambient temperature, in particular cyclopolydimethylsiloxanes (cyclomethicones) e.g. cyclohexasiloxane and cyclopentasiloxane; polydimethylsiloxanes (or dimethicones) having alkyl, alkoxy or phenyl groups pendant or at the end of the silicone chain, groups having 2 to 24 carbon atoms; phenylated silicones such as phenyltrimethicones (in particular a diphenylsiloxyphenyltrimethicone), phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, polymethylphenylsiloxanes, and mixtures thereof.

In one particular embodiment, the content of vegetable oil(s) in the fatty phase of the droplets (G1) of an emulsion of the invention is between 0% and 40%, preferably between 0.1% and 25%, and in particular between 1% and 20% by weight relative to the total weight of said fatty phase of the droplets (G1).

In one embodiment, the emulsions of the invention comprise from 0% to 99.49%, preferably from 5% to 95%, in particular from 20% to 90%, and better still from 30% to 80%, even 50% to 70% by weight of oil(s) relative to the total weight of the fatty phase of the droplets (G1).

Droplets (G2)

As indicated above, each droplet (G1) comprises at least one droplet (G2) comprising the internal aqueous phase. Preferably, each droplet (G1) comprises a single droplet (G2) comprising the internal aqueous phase.

In one particular embodiment, the internal phase of the droplets (G2) of a dispersion of the invention is a gas phase. For example, the internal phase of said droplets (G2) comprises at least one gas selected for example from among air, oxygen, nitrogen, nitrous oxides, rare gases, carbon dioxide and mixtures thereof.

A droplet (G2) of the invention is composed of a core, also called inside of the droplet. Optionally a droplet (G2) of the invention is surrounded by a shell to insulate the inside of the droplet from the fatty phase of the emulsion.

In one embodiment, the size of the droplets (G2) is greater than 10 μm, even greater 50 μm, better still it is between 10 μm and 2 000 μm, in particular between 50 μm and 1 500 μm, better still between 100 μm and 1 100 μm, in particular between 200 μm and 800 μm, and better still between 300 μm and 700 μm.

In particular, an emulsion of the invention comprises from 0.01% to 50%, preferably 0.1% to 40%, in particular 1% to 30%, better still from 2.5% to 20% by weight of droplets (G2) relative to the total weight of said emulsion.

In one embodiment, and as previously indicated, an emulsion of the invention is obtained with a microfluidic method such as defined below. The droplets (G2) therefore have uniform size distribution. Preferably, the internal aqueous phase of the emulsions of the invention is composed of a population of monodisperse droplets (G2), in particular such that they have a mean diameter D of between 10 μm and 2 000 μm and a coefficient of variation Cv lower than 10%, even lower than 3% measured using the above-described methods.

Preferably, the droplets (G1) and (G2) are respectively monodisperse droplets, such as defined above. For obvious reasons, for a given population of droplets (G1), the mean diameter of droplets (G1) is greater than the mean diameter of droplets (G2).

In one embodiment, in the emulsions of the invention, the volume fraction p (IF/(IF+MF) is between 0.1 and 0.7, preferably between 0.3 and 0.6, better still between 0.4 and 0.5, where:
IF is the total volume of droplets (G2), and
MF is the total volume of droplets (G1) (therefore without the total volume of droplets (G2)).

In one embodiment, an emulsion of the invention may comprise at least two populations of droplets (G1) differing from each other in particular through the diameter of the droplets (G1) and/or the type of raw materials of the droplets (G1) and/or the content of the raw materials of the droplets (G1) and/or the diameter of the droplets (G2) and/or the type of raw materials of droplets (G2) and/or the raw material content of droplets (G2).

By «raw materials» it is meant to designate any type of compound able to be used in the fatty phase of droplets (G1) and the internal aqueous phase of droplets (G2), in particular cationic polymers, anionic polymers, oils, gelling agents, texturing agents, active substances and additional compounds described herein.

Shell of Droplets (G1) and Optionally of Droplets (G2)

As previously mentioned, droplets (G1), and optionally droplets (G2), of the invention are surrounded by a shell (also designated by the term «membrane»).

In the invention, droplets (G1), and optionally droplets (G2) obtained may have a very thin shell having a thickness in particular of less than 1% of the diameter of the droplets.

The thickness of the shell is therefore preferably thinner than 1 μm and is too small to be measured using optical methods.

In one embodiment, the thickness of the shell of droplets (G1), and optionally of droplets (G2), is less than 1 000 nm, and in particular lies between 1 and 500 nm, preferably less than 100 nm, advantageously less than 50 nm, preferably less than 10 nm, and more particularly it is 100 nm a 300 nm.

Measurement of the shell thickness of droplets (G1), and optionally of droplets (G2), of the invention can be carried out using Small-Angle X-ray Scattering, such as used in Sato et al. J. Chem. Phys. 111, 1393-1401 (2007).

For this measurement, the droplets are produced using deuterated water then washed three times with deuterated oil e.g. a deuterated oil of hydrocarbon type (octane, dodecane, hexadecane).

After washing, he droplets are transferred to the Neutron cell to determine the spectrum 1(q); q being the wave vector.

From this spectrum, conventional analytical processing is applied (REF) to determine the thickness of the hydrogenated (non-deuterated) shell.

In one embodiment, the shell surrounding droplets (G1), and optionally droplets (G2), is rigidified, in particular to impart good droplet strength and to reduce, even prevent, their coalescence.

This shell is typically formed by coacervation, i.e. by precipitation of polymers having opposite charges. Within a coacervate, the bonds linking together the charged polymers are of ionic type, and are generally stronger than the bonds contained within a membrane of surfactant type.

The shell is formed by coacervation of at least two polymers having charges of opposite polarity (or polyelectrolyte) and preferably in the presence of a first polymer of cationic type and a second polymer differing from the first polymer, of anionic type. These two polymers act as membrane-rigidifying agents.

The formation of the coacervate between these two polymers is generally caused by modifying the conditions of the reaction medium (temperature, pH, reagent concentration, etc.). The coacervation reaction results from neutralisation of these two polymers having charges of opposite polarity, and allows the formation of a membrane structure via electrostatic interactions between the anionic polymer and cationic polymer. The membrane thus formed around each droplet typically forms a shell which fully encapsulates the core of the droplet and therefore insulates the core of the droplet from the continuous aqueous phase.

Anionic Polymers (PA1) and Optionally (PA2)

The external aqueous phase comprises at least one anionic polymer (PA1) and optionally the internal aqueous phase also comprises at least one anionic polymer (PA2). When the internal aqueous phase also comprises at least one anionic polymer (PA2), the polymers (PA1) and (PA2) can be the same or different.

Preferably, the anionic polymer(s) (PA1), even the anionic polymer(s) (PA2) if any are hydrophilic i.e. soluble or dispersible in water.

In the present description, by “anionic polymer” (or “polymer of anionic type”) is meant a polymer comprising chemical functions of anionic type. The term anionic polyelectrolyte can also be used.

By “chemical function of anionic type” is meant a chemical AH function capable of giving away a proton to yield a function A⁻. Depending on the conditions of the medium in which it is contained, the polymer of anionic type therefore comprises chemical functions in AH form, or else in the form of its conjugate base A⁻.

As examples of chemical functions of anionic type, mention can be made of carboxylic acid functions —COOH, optionally present in the form of a carboxylate anion —COO—.

As examples of polymers of anionic type, mention can be made of any polymer formed by polymerization of monomers of which at least one portion carries chemical functions of anionic type, such as carboxylic acid functions. For example, such monomers are acrylic acid, maleic acid or any ethylenically saturated monomer having at least one carboxylic acid function. Therefore, preferably, the anionic polymers (PA1) and (PA2), the same or different, are polymers having monomeric units comprising at least one carboxylic acid function.

Among the examples of anionic polymers suitable for use in the invention, mention can be made of copolymers of acrylic acid or maleic acid and other monomers, such as acrylamide, alkyl acrylates, C₅-C₈ alkyl acrylates, C₁₀-C₃₀ alkyl acrylates, C₁₂-C₂₂ alkyl methacrylates, methoxypolyethyleneglycol methacrylates, hydroxyester acrylates, crosspolymer acrylates, and mixtures thereof.

In one embodiment, the anionic polymers of the invention are selected from among carbomers, and acrylates/C₁₀₋₃₀ alkyl acrylate crosslinked copolymers. Preferably the anionic polymers (PA1) and (PA2) of the invention are carbomers.

In one embodiment, the shell of droplets (G1) comprises at least one anionic polymer (PA1), e.g. a carbomer.

In one embodiment, the internal aqueous phase also comprises at least one anionic polymer (PA2), which means that the shell of droplets (G2) comprises at least one anionic polymer (PA2) e.g. a carbomer.

In the invention, and unless otherwise indicated, by “carbomer” is meant an optionally crosslinked homopolymer derived from polymerization of acrylic acid. It is therefore a polyacrylic acid, optionally crosslinked.

Among the carbomers of the invention, mention can be made of those marketed under the trade names Tego® Carbomer 340FD by Evonik, Carbopol® 981 by Lubrizol, Carbopol ETD 2050 by Lubrizol, or Carbopol Ultrez 10 by Lubrizol.

In one embodiment, by “carbomer” or “Carbopol®” is meant an acrylic acid polymer of high molecular weight crosslinked with allylic sucrose or allylic ethers of pentaerythritol (Handbook of Pharmaceutical Excipients, 5th Edition, pill). For example, it is Carbopol®10, Carbopol®934, Carbopol®934P, Carbopol®940, Carbopol®941, Carbopol®71G, Carbopol®980, Carbopol®971P or Carbopol®974P. In one embodiment the viscosity of said carbomer is between 4 000 and 60 000 cP at 0.5% w/w.

Carbomers have other names: polyacrylic acids, carboxy vinyl polymers or carboxy polyethylenes.

In the invention, the anionic polymer (PA1), and optionally anionic polymer (PA2), can also be an acrylates/C₁₀₋₃₀ alkyl acrylate crosslinked polymer (INCI name: acrylates/C₁₀₋₃₀ alkyl acrylate Crosspolymer) such as defined above.

In the invention, the emulsions of the invention may comprise a carbomer and acrylates/C₁₀₋₃₀ alkyl acrylate Crosspolymer

In one embodiment, an emulsion of the invention comprises from 0.1% to 5%, preferably 0.05% to 2%, and better still 0.1% to 0.5% by weight of polymer (PA1) relative to the total weight of said emulsion.

In one embodiment, when droplets (G2) also comprise a shell such as previously described, an emulsion of the invention comprises from 0.001% to 0.5%, preferably 0.005% to 0.5%, and better still 0.01% to 0.1% by weight of polymer (PA2) relative to the total weight of said emulsion.

In the invention, the above-mentioned emulsion may comprise from 0.01% to 5%, preferably 0.05% to 2%, and more preferably 0.1% to 0.5% by weight of anionic polymer(s) (PA1), and optionally of anionic polymer(s) (PA2), carbomer(s) in particular, relative to the total weight of said emulsion.

Cationic Polymer

The dispersed fatty phase comprises at least one cationic polymer (PC).

Therefore, droplets (G1), and particularly the shell of said droplets (G1), even optionally the shell of droplets (G2), also comprise at least one polymer of cationic type. They may also comprise several polymers of cationic type. This cationic polymer is the one mentioned above forming the shell by coacervation with the anionic polymer.

In the present application, and unless otherwise indicated, by “cationic polymer” (or “polymer of cationic type”) is meant a polymer comprising chemical functions of cationic type. The term cationic polyelectrolyte can also be used.

Preferably, the cationic polymer (PC) is lipophilic or liposoluble.

In the present application, and unless otherwise indicated, by “chemical function of cationic type”, is meant a chemical function B capable of capturing a proton to give a function BH⁺. Depending on the conditions of the medium in which it is contained, the polymer of cationic type therefore comprises chemical functions in B form or else in BH⁺ form, its conjugate acid.

As examples of chemical functions of cationic type, mention can be made of primary, secondary and tertiary amine functions, optionally present in the form of ammonium cations.

As examples of cationic polymers, any polymer can be cited that is formed by polymerization of monomers of which at least one portion carries chemical functions of cationic type, such as primary, secondary or tertiary amine functions.

For example, said monomers are aziridine, or any ethylenically unsaturated monomer comprising at least one primary, secondary or tertiary function.

Among the examples of cationic polymers suitable for implementing the invention, mention can be made of amodimethicone, derived from a silicone polymer (polydimethylsiloxane, also called dimethicone), modified by primary amine and secondary amine functions.

It is also possible to cite derivatives of amodimethicone, e.g. copolymers of amodimethicone, aminopropyl dimethicone, and more generally straight-chain or branched silicone polymers comprising amine functions.

Mention can be made of the copolymer of bis-isobutyl PEG-14/amodimethicone, Bis (C13-15 Alkoxy) PG-Amodimethicone, Bis-Cetearyl Amodimethicone and bis-hydroxy/methoxy amodimethicone.

Mention can also be made of polymers of polysaccharide type comprising amine functions such as chitosan or the derivatives of guar gum (guar hydroxypropyltrimonium chloride).

The polymers of polypeptide type can also be cited comprising amine functions, such as polylysine.

The polymers of polyethyleneimine type can also be cited comprising amine functions, such as straight-chain or branched polyethyleneimine.

In one embodiment, the droplets and in particular the shell of said droplets comprise a cationic polymer which is a silicone polymer modified by a primary, secondary or tertiary amine function, such as amodimethicone.

In one embodiment, the droplets and in particular the shell of said droplets comprise amodimethicone.

In one particularly preferred embodiment, the cationic polymer(s) (PC) meet following formula (I):

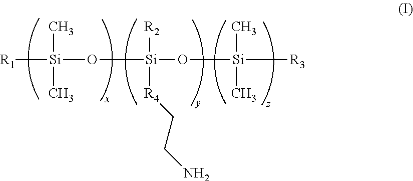

where:
R₁, R₂ and R₃ are each independently OH or CH₃;
R₄ is a —CH₂— group or —X—NH— group where X is a C₃ or C₄ divalent alkylene radical;
x is an integer between 10 and 5 000, preferably between 30 and 1 000, better still between 80 and 300;
y is an integer between 2 and 1 000, preferably between 4 and 100, better still between 5 and 20; and
z is an integer between 0 and 10, preferably between 0 and 1, and better still it is 1.

In above-mentioned formula (I), when R₄ is a —X—NH— group, X is linked to the silicon atom.

In above-mentioned formula (I), R₁, R₂ and R₃ are preferably CH₃.

In above-mentioned formula (I), R₄ is preferably a —(CH₂)₃—NH— group.

An amodimethicone differs from an oil such as those previously described and able to form the fatty phase of droplets (G1) of an emulsion of the invention.

In the invention, the emulsion may comprise from 0.01% to 10%, preferably 0.05% to 5% by weight of cationic polymer(s) (PC), amodimethicone(s) in particular, relative to the total weight of the fatty phase of droplets (G1).

Internal Aqueous Phase

As previously indicated, droplets (G2) of the invention comprise an internal aqueous phase.

In one embodiment, this aqueous phase has viscosity of between 0 mPa·s and 10 000 mPa·s, preferably between 0 mPa·s and 2 000 mPa·s, such as measured at 25° C.

This viscosity is measured with the method described above.

The internal aqueous phase of the dispersions at least comprises water.

In addition to distilled or deionized water, one water suitable for the invention can also be a naturally sourced water or floral water.

In one embodiment, the weight percent of water in the internal aqueous phase is at least 30%, preferably at least 40%, in particular at least 50%, better still at least 60%, in particular between 70% and 98%, and preferably between 75% and 95%, relative to the total weight of said internal aqueous phase.

In one particular embodiment, the external continuous aqueous phase and/or internal aqueous phase can be in the form of an oil-in-water emulsion, the same or different, said emulsion comprising a continuous aqueous phase and a dispersed fatty phase in the form of droplets (G3), the size of droplets (G3) being less than 500 μm, preferably less than 400 μm, in particular less than 300 μm, better still less than 200 μm, in particular less than 100 μm, even less than 20 μm, and better still less than 10 μm. Preferably the size of droplets (G3) is between 0.1 and 200 μm, preferably between 0.25 and 100 μm, in particular between 0.5 μm and 50 μm, preferably between 1 μm and 20 μm, better still between 1 μm and 10 μm, even between 3 μm and 5 μm.

Optionally, droplets (G3) comprise a shell formed of at least one anionic polymer, the same as or differing from the anionic polymers (PA1) and/or (PA2), and at least one cationic polymer, the same as or differing from the cationic polymer (PC),

In another particular embodiment, the fatty phase of droplets (G1) can be in the form of a water-in-oil emulsion, said emulsion comprising a continuous fatty phase and dispersed aqueous phase in the form of droplets (G4), the size of droplets (G4) being smaller than that of droplets (G1) and preferably smaller than droplets (G2). Preferably, the size of the droplets (G4) is less than 500 μm, preferably less than 400 μm, in particular less than 300 μm, better still less than 200 μm, and in particular less than 100 μm, even less than 20 μm, and better still less than 10 μm. Preferably, the size of droplets (G4) is between 0.1 and 200 μm, preferably between 0.25 and 100 μm, in particular between 0.5 μm and 50 μm, preferably between 1 μm and 20 μm, and better still between 1 μm and 10 μm, even between 3 μm and 5 μm.

Optionally, droplets (G4) comprise a shell formed of at least one anionic polymer, the same as or differing from anionic polymer (PA1), and at least one cationic polymer, the same as or differing from cationic polymer (PC).

Advantageously, droplets (G3) and/or (G4) are not macroscopic i.e. non-visible to the naked eye.

In other words, droplets (G3) and/or (G4) are different and independent of droplets (G1) and (G2). Also, when droplets (G3) are contained in the internal aqueous phase, the size of droplets (G3) is smaller than the size of droplets (G2).

These droplets (G3) and/or (G4) of reduced size allow an effect on texture. An emulsion of the invention comprising said finely dispersed droplets (G3) and/or (G4) has qualities of smoothness that are further enhanced.

The presence of droplets (G3) and/or (G4) reinforces the characteristics of an emulsion in terms of unique texture, lightweight and evolving sensory aspect. More particularly, an emulsion of the invention comprising droplets (G3) and/or (G4) spreads easily over the skin. The first instants of application are highly aqueous with marked breakdown effect. The feeling then progresses towards an oily veil which fades leaving a lightweight, hydrated skin. This texture is particularly advantageous and surprising for skilled persons in the light of the fact that there are no surfactants in these emulsions.

Texturing Agent(s)

Depending on the fluidity and/or sensory effect and/or texture of the emulsion of the invention that it is desired to obtain, said emulsion and in particular the external aqueous phase and/or internal aqueous phase may also comprise at least one texturing agent differing from the previously described cationic polymers, anionic polymers, oils and gelling agents.

Evidently, persons skilled in the art will take care to select any optional texturing agent(s) and/or the amounts thereof so that the advantageous properties of an emulsion of the invention are not or are not substantially affected by the envisaged addition.

Advantageously, an emulsion of the invention comprises from 0.01% to 50%, preferably 0.05% to 30%, in particular 0.1% to 15%, better still 1% to 10%, and more particularly 2% to 5%, by weight of texturing agent relative to the total weight of said emulsion.

In particular, when the external aqueous phase comprises at least one texturing agent, an emulsion of the invention comprises from 0.01% to 50%, preferably 0.05% to 30%, in particular 0.1% to 15%, better still 1% to 10%, and most particularly 2% to 5% by weight of texturing agent(s) relative to the total weight of said external aqueous phase.

In particular, when the internal aqueous phase comprises at least one texturing agent, an emulsion of the invention comprises from 0.01% to 30%, preferably 0.05% to 15%, in particular 0.1% to 10% by weight of texturing agent(s) relative to the total weight of said internal aqueous phase.

As hydrophilic texturing agents i.e. soluble or dispersible in water and therefore able to be contained in the external aqueous phase and/or internal aqueous phase of an emulsion of the invention, mention can be made of:
natural texturing agents selected in particular from among algae extracts, plant exudates, seed extracts, microorganism exudates such as alcasealan (INCI: Alcaligenes Polysaccharides), and other natural agents, in particular hyaluronic acid;
semi-synthetic texturing agents selected in particular from among cellulose derivatives and modified starches;
synthetic texturing agents selected in particular from among homopolymers of methacrylic acid or one of the esters thereof, the copolymers of methacrylic acid or one of the esters thereof, AMPS copolymers (2-acrylamido-2-methylpropane sulfonic acid), associative polymers;
other texturing agents selected in particular from among glycols, polyethylene glycols (marketed under the trade name Carbowax), clays, silicas such as those marketed under the trade names Aerosil® 90/130/150/200/300/380), in particular glycerine, propylene glycol, butylene glycol, penthylene glycol, propanediol, methylpropanediol, hexanediol, and
mixtures thereof.

By «associative polymer» in the meaning of the present invention is meant any amphiphilic polymer having in its structure at least one fatty chain and a least one hydrophilic portion; associative polymers conforming to the present invention can be anionic, cationic, nonionic or amphoteric; in particular they are those described in FR 2 999 921. Preferably, they are amphiphilic and anionic associative polymers, and amphiphilic and nonionic associative polymers such as described below.

Preferably, the texturing agents of the aqueous phase are selected from among those resistant to electrolytes, and selected in particular from among carrageenans, xanthan gum; carboxymethylcellulose; hydroxyethylcellulose; hydroxypropyl cellulose; hydroxypropyl methylcellulose; methylcellulose; ethylcellulose; alkylhydroxyethyl celluloses; hydroxypropyl starch phosphate; carbomers among those marketed under the trade names Carbopol Ultrez 10/30, Tego Carbomer 134/140/141, Aqupec HV-505, HV-505HC, HV-504, HV-501, HV-505E, HV-504E, HV-501E, HV-505ED, Ashland 941 carbomer or Ashland 981 carbomer; acrylate copolymers particularly those marketed under the trade names Carbopol Aqua SF-1 Polymer, or Carbopol Aqua SF-1 OS Polymer, or Arkema Reostyl 67N; acrylates/C10-C30 alkyl acrylate crosspolymers marketed under the trade names Carbopol Ultrez 20/21,Tego Carbomer 341 ER, Tego Carbomer 750 HD, Tego Carbomer 841 SER, Aqupec HV-501 ER, HV-701 EDR, HV-501 EM, SER W-150C or SER W-300C; sodium acrylates/beheneth-25 methacrylate crosspolymer; acrylates/acrylamide copolymers; AMPS Na/hydroxyethyl acrylate copolymers marketed under the trade names Sepinov WEO or Sepinov EMT 10; acryloyl Dimethyltaurate/Sodium Acrylate/Dimethylacrylamide crosspolymers; PVP; acrylates/Steareth-20 Methacrylate Copolymer; Polyacrylate Crosspolymer-6; acrylates/ceteth-20 itaconate copolymer; polyurethanes e.g. Steareth-100/PEG-136/HDI Copolymer marketed under the trade name RHEOLUXE® 811 by Elementis specialitis, in particular polyether polyurethanes such as PEG-240/HDI COPOLYMER BIS-DECYLTETRADECETH-20 ETHER, particularly those marketed under the trade names Adeka Nol GT-700/GT-730; polyurethane-39; cetyl hydroxyethylcellulose; polyethylene glycols; bentonite; glycerine; and mixtures thereof, and better still selected from among acrylates copolymers in particular the one marketed under the trade name Carbopol Aqua SF-1 Polymer or Carbopol Aqua SF-1 OS Polymer.

These texturing agents, in addition to their electrolyte-resistant properties, impart improved stability and transparency to an emulsion of the invention containing the same.

Additional Compound(s)

In the invention, the internal aqueous phase and/or external aqueous phase and/or fatty phase can further comprise at least one additional compound differing from the above-mentioned anionic and cationic polymers, gelling agent, texturing agent and oils

An emulsion of the invention, and in particular the internal aqueous phase and/or external aqueous phase and/or fatty phase of said emulsion may further comprise as additional compound: powders, flakes, colouring agents particularly selected from among those that are or are not water-soluble, liposoluble or not, organic or inorganic, pigments, materials having optical effect, liquid crystals and mixtures thereof, particulate agents insoluble in the fatty phase, emulsifying and/or non-emulsifying silicone elastomers in particular such as described in EP 2 353 577, preserving agents, humectants, stabilizers, chelating agents, emollients, modifying agents selected from among pH agents, osmotic pressure agents and/or refractive index modifiers etc. . . . or any usual cosmetic additive, and mixtures thereof.

In one embodiment, the internal aqueous phase and/or external aqueous phase comprises at least one colouring agent such as defined above. Preferably, the internal aqueous phase of the emulsions of the invention comprises at least one colouring agent.

The emulsions of the invention and in particular the internal aqueous phase and/or external aqueous phase and/or fatty phase of the emulsions, may further comprise at least one active substance, biologic or cosmetic in particular, preferably selected from among hydrating agents, healing agents, depigmenting agents, UV filters, peeling agents, antioxidants, active substances stimulating the synthesis of dermal and/or epidermal macromolecules, dermo-relaxants, anti-perspirants, soothing agents, anti-age agents, fragrances and mixtures thereof. Said active substances are notably described in FR 1 558 849.

In one embodiment, the additional compound(s) or active substance(s), particularly a hydrophilic cosmetic active substance added to an internal aqueous phase of an emulsion of the invention, preferably have a Log P lower than 1, in particular lower than 0.5, more preferably lower than 0, even between 0.5 and −2.5, and better still between 0 and −2.5.

In one embodiment, the additional compound(s) and/or active substance(s), in particular a lipophilic cosmetic active substance added to the fatty phase of droplets (G1) of an emulsion of the invention, preferably have a Log P higher than 1, more preferably higher than 2, better still higher than 3, even between 1 and 7, in particular between 1.5 and 5, and better still between 2 and 3.5.

Log P (octanol-water partition coefficient of a molecule) gives an estimation of the hydrophobia of a molecule under consideration and has the advantage of being referenced/tabulated and hence of being easily accessible for most conventional molecules. In addition, the value of Log P (=log (K)) can be evaluated simply using molecule modelling software easily accessible on the internet e.g. at www.molispiration.com, www.vcclab.org/lab/alogps/start.html . . .

Experimental determination is possible using the following method: a precise amount of active substance is weighed and then solubilized in one of the two phases, water or octanol. Two equivalent volumes of the 2 phases are then placed in contact under agitation. The concentrations of the active substance in each of the two phases are then determined after thermodynamic equilibration of the system. This measurement of concentration can be performed for example via direct measurement of absorbance, if the molecule absorbs light, or via liquid chromatography. This measurement is performed for example at 22° C.

Coefficient K is then determined experimentally with the ratio of the concentration of active substance in octanol to the concentration in water.

In one embodiment, an emulsion of the invention is such that the fatty phase of droplets (G1) also comprises at least one lipophilic (or liposoluble) active substance, and the internal aqueous phase also comprises at least one hydrophilic (or water-soluble) active substance.

In one embodiment, the emulsions of the invention comprise glycerine in the internal aqueous phase and/or external aqueous phase. Preferably, the emulsions of the invention comprise at least 5 weight % of glycerine relative to the total weight of said emulsions. In addition to texture, the emulsions of the invention provide another advantage compared with «conventional» emulsions since they allow the use of glycerine and in high contents.

In particular, they may comprise glycerine in an amount of 10% or higher, 20% or higher, 30% or higher, 40% or higher, even up to 50% by weight relative to the total weight of the emulsions.

In one particular embodiment, an emulsion of the invention is such that the fatty phase of droplets (G1) also comprises at least one colouring agent (C1), and the internal aqueous phase of droplets (G2) also comprises at least one colouring agent (C2), (C2) differing from (C1) in particular with regard to colour effect. Preferably, the colouring agents (C1) and (C2) are selected from among pigments, pearl additives and mixtures thereof.

This embodiment is advantageous in that the colour effect obtained when applying an emulsion of the invention onto keratinous material differs from the colour displayed by said emulsion before application. Before application of the emulsion, and hence before rupture of droplets (G1) and (G2), the colour effect mostly seen—even the sole visible colour effect is the one shown by droplets (G1).

Application of an emulsion of the invention onto keratinous material leads (i) to revealing the colour effect of droplets (G2) and hence (ii) to a new unexpected colour effect derived from the mixture of colouring agents (C1) and (C2).

In another particular embodiment, an emulsion of the invention is such that the fatty phase of droplets (G1) also comprises at least one UV filter, and the internal aqueous phase of droplets (G2) also comprises at least one active substance, biological or cosmetic in particular, differing from the UV filter, and in particular an active substance sensitive to (or unstable under) sun radiation and more particularly under UV.

This embodiment is advantageous in that the presence of UV filters in the fatty phase of droplets (G1) provides protection to the active substance contained in the internal aqueous phase of droplets (G2) against the effects of sun radiation and notably of UV rays. Therefore, the integrity of said active substance can be preserved over longer time periods. This is of particular advantage for active substances sensitive to sun radiation such as vitamin B, vitamin C, dihydroxyacetone or DHA, EUK 134 (INCI name: Ethylbisiminomethylguaiacol manganese chloride), etc. . . .

Advantageously, when an emulsion of the invention, in particular the fatty phase of droplets (G1), also comprises at least one fragrance, the external aqueous phase, even also the internal aqueous phase, further comprise at least one crosslinked polymer of crosslinked copolymer differing from the anionic polymer, in particular differing from the carbomer(s) and/or acrylates/C10-30 alkyl acrylate crosspolymers mentioned above, said crosslinked polymer or crosslinked copolymer comprising at least one unit derived from the polymerization of one of the monomers selected from the group formed by acrylic acid, methacrylic acid, alkyl acrylate having 1 to 30 atoms.

In the invention, and unless otherwise indicated, by «crosslinked copolymer of methacrylic acid and alkyl acrylate having 1 to 4 carbon atoms» is meant a crosslinked copolymer resulting from the polymerization of a monomer of methacrylic acid and a monomer of alkyl acrylate having 1 to 4 carbon atoms.

In one embodiment, the crosslinked polymer or crosslinked copolymer of the invention contained in the continuous aqueous phase, is selected from the group formed by the following polymers or copolymers: Acrylates Copolymer, Acrylates crosspolymer-4, Acrylates crosspolymer-3, Polyacrylate-2 Crosspolymer and Polyacrylate-14 (INCI names).

Among said above polymers, and most particularly according to the invention, preference is given to the products sold by LUBRIZOL under the trade names (INCI name=Polyacrylate-2 Crosspolymer), Fixate Freestyle Polymer (INCI name=Acrylates crosspolymer-3), Carbopol® Aqua SF1 (INCI name=Acrylates copolymer) and Carbopol® Aqua SF2 (INCI name=Acrylates crosspolymer-4), or the product sold by Croda Inc. under the trade name Volarest™ FL.

Preferably, the crosslinked polymer or crosslinked copolymer are selected from among Carbopol® Aqua SF1 (INCI name=Acrylates copolymer) and Carbopol® Aqua SF2 (INCI name=Acrylates crosspolymer-4).

In one embodiment, the crosslinked copolymer is selected from among the crosslinked copolymers of acrylic or methacrylic acid and alkyl acrylates having 1 to 4 carbon atoms.

In the invention, the emulsion may comprise from 0.1% to 10% by weight, preferably 0.5% to 8% by weight, and preferably 1% to 3% by weight of crosslinked polymer(s) or crosslinked copolymer(s) relative to the total weight of said emulsion.

Additionally, when an emulsion of the invention in particular the fatty phase of droplets (G1), also comprises at least one fragrance, the external aqueous phase, even internal aqueous phase, may also comprise at least one buffer having a pKa of between 4.0 and 9.0, selected in particular from the group formed by phosphate buffers, 2-(N-morpholino)ethane sulfonic acid, 2-amino-2-hydroxymethyl-1,3-propanediol, 2-(bis(2-hydroxyethyl)amino)acetic acid, 4-(2-hydroxyethyl)-1-piperazine ethane sulfonic acid, sodium citrate and mixtures thereof, preferably 4-(2-hydroxyethyl)-1-piperazine ethane sulfonic acid. Preferably, an emulsion of the invention comprises from 0.1% to 10% by weight of buffer(s), preferably 0.5% to 5% by weight relative to the total weight of said emulsion.

Evidently, those skilled in the art will take care to select any above-mentioned additional compound(s) and/or active substance(s) and/or respective amounts thereof so that the advantageous properties of the emulsion of the invention are not or are not substantially altered by the envisaged addition. In particular, the type and/or amount of the additional compound(s) or active substance(s) are dependent on the aqueous or fatty nature of the phase under consideration of the emulsion of the invention. These adjustments are within the reach of skilled persons.

Preparation Method

The present invention also concerns a method for preparing an emulsion such as defined above, comprising the following steps:

a) placing an aqueous fluid FE1 in contact with an oily fluid F1;

b) forming a water-in-oil emulsion composed of droplets (G2), formed by aqueous fluid FE1, dispersed in a fatty phase formed by fluid F1; and

c) forming droplets (G1), each droplet (G1) being composed of at least one, preferably a single droplet (G2), by contacting the water-in-oil emulsion obtained at step b) with an aqueous fluid FE2;

wherein:
the aqueous fluid FE1 at least comprises water and optionally at least one anionic polymer (PA2), in particular a carbomer;
the oily fluid F1 comprises at least one gelling agent and at least one cationic polymer (PC), in particular an amodimethicone, and optionally at least one oil; and
the aqueous fluid FE2 at least comprises water and at least one anionic polymer (PA1), in particular a carbomer the same as or differing from the anionic polymer (PA2).

In one embodiment, above-mentioned steps b) and c) can be simultaneous.

An emulsion of the invention is preferably obtained with a microfluidic method i.e. by using a microfluidic device such as described in WO 2012/120043.

In one embodiment, step c) above may also comprise the presence of an intermediate fluid miscible with fluid F1, as described in WO 2012/120043. At the formation step of droplets (G1), this intermediate fluid is therefore present at the interface between fluid F1 and fluid FE2. This intermediate fluid is intended to form a film around droplet (G1) in formation at the microfluidic device. The intermediate fluid differs from the fatty phase of droplets (G1) at least in that it is devoid of cationic polymer. Therefore, the intermediate fluid delays diffusion of the cationic polymer (PC) contained in fluid F1 until the intermediate fluid has mixed with fluid F1 thereby ensuring the formation of very stable droplets, stabilized by a very thin shell without obstructing the microfluidic device.

If the aqueous fluid FE1 also comprises an anionic polymer (PA2), step a) above may further comprise the presence of an intermediate fluid miscible with fluid F1, as described in WO 2012/120043. At the formation step of droplets (G2), this intermediate fluid is therefore present at the interface between fluid F1 and fluid FE1. This intermediate fluid is intended to form a film around droplet (G2) being formed at the microfluidic device. Therefore, the intermediate fluid delays diffusion of the cationic polymer (PC) contained in fluid F1 until the intermediate fluid has mixed with fluid F1 thereby ensuring the formation of very stable droplets (G2), stabilized by a very thin shell without obstructing the microfluidic device.

In another embodiment, a method of the invention may further comprise a step d) to add a viscosity-enhancing solution to the external aqueous phase namely fluid FE2. Preferably, the viscosity-enhancing solution is therefore aqueous. This viscosity-enhancing solution is typically added to the aqueous fluid FE2 after formation of droplets (G1) and (G2), step d) therefore following after step c). In one embodiment, the viscosity-enhancing solution comprises a base, in particular an alkaline hydroxide such as sodium hydroxide.

In yet another embodiment, when the gelling agent contained in the oily fluid F1 is a heat-sensitive gelling agent such as previously described, the method for preparing an emulsion of the invention may require the use at least of fluid F1 at a temperature of 40° C. to 150° C.

Therefore, in this embodiment, at least fluid F1, even aqueous fluid FE1, can be heated to a temperature of 40° C. to 150° C. to perform step a), even steps b) and c), and optionally fluid FE2 can be heated to a temperature of 40° C. to 150° C. to perform step c).

If the method for preparing an emulsion of the invention is a microfluidic method, the microfluidic device as such is advantageously heated to a temperature of 40° C. to 150° C.

Preferably, the heating temperature of fluid F1, even of fluids FE1 and FE2, and of the microfluidic device is 50° C. to 100° C., preferably 55° C. to 90° C., more preferably 60° C. to 80° C., and further preferably 65° C. to 85° C.

In one embodiment, when the oily fluid F1 comprises 5 to 15 weight % of heat-sensitive gelling agent(s) relative to the total weight of said oily fluid F1, said oily fluid is preferably heated to a temperature of 65° C. to 70° C.

In one embodiment, when the oily fluid F1 comprises 15% to 99.99%, preferably 15% to 40% by weight of heat-sensitive gelling agent(s) relative to the total weight of said oily fluid F1, said oily fluid F1 is preferably heated to a temperature of 80° C. to 90° C.

Advantageously, the presence of a gelling agent in oily fluid F1 obviates the use of an intermediate fluid such as described in application WO 2012/120043. In this respect, the method for preparing a dispersion according to the invention is simplified compared with the preparation method described in WO 2012/120043.

Preferably, the microfluidic device used in the invention comprises one or more of the following characteristics taken alone or in any technically possible combination:
the outlet orifices of the different lines (or paths or channels) of the microfluidic device preferably lie on one same horizontal axis, so that each droplet (G1) and associated internal droplet (G2) are formed simultaneously (in other words, the ends of the IF and MF channels are placed at the same height so that the droplets are formed in a single step). This embodiment is advantageous in that it provides control over the number of droplets (G2) in each droplet (G1), and in particular can ensure the presence of a single droplet (G2) in each droplet (G1);
at the outlet of the microfluidic device, the different fluids employed form a multi-component droplet, in so-called «dripping» hydrodynamic mode.

Utilizations

Preferably an emulsion of the invention can be used directly after the aforementioned preparation methods, as a composition in particular a cosmetic composition. A composition of the invention, when prepared with a microfluidic method such as described above, can also be used as composition, particularly a cosmetic composition, after separation of droplets (G1) and redispersion thereof in a second suitable phase.

The compositions of the invention can notably be used in the cosmetic field.

In addition to the above-mentioned ingredients, they may comprise at least one physiologically acceptable medium.

By “physiologically acceptable medium” it is meant to designate a medium particularly suitable for application of a composition of the invention onto keratinous material, in particular the skin, lips, nails, eyelashes, eyebrows, and preferably the skin.

The physiologically acceptable medium is generally adapted to the site of intended application of the composition, and to the intended presentation of the packaged composition.

In one embodiment, the physiologically acceptable medium is determined directly by the external aqueous phase such as described above.

In one embodiment, the cosmetic compositions are used for make-up and/or care of keratinous material, the skin in particular.

The cosmetic compositions of the invention can be care products, sun protection, cleansing (makeup removal) products, hygiene or makeup products for the skin.

These compositions are therefore intended to be applied to the skin in particular.

The present invention therefore also concerns the non-therapeutic, cosmetic use of an above-mentioned cosmetic composition as makeup, hygiene, cleansing product and/or care product for keratinous material, the skin in particular.

In one embodiment, the compositions of the invention are in the form of a foundation, makeup remover, face and/or body and/or hair care product, an anti-age product, sun protection, care products for greasy skin, whitening, hydration, BB cream, tinted cream or foundation, facial and/or body cleanser, shower gel or shampoo.

A care composition of the invention in particular can be a sun composition, care cream, serum or deodorant.

The compositions of the invention can be in various forms, particular in the form of a cream, balm, lotion, serum, gel, gel-cream, or mist spray.

The present invention also concerns a non-therapeutic method for the cosmetic treatment of keratinous material, the skin in particular, comprising at least one step to apply to said keratinous material at least one emulsion or composition of the invention.

In particular, the present invention concerns a non-therapeutic method for cosmetic treatment of the skin, comprising a step to apply to the skin at least one emulsion or composition of the invention.

The present invention also concerns the use of a water-in-oil-in-water emulsion comprising an external continuous aqueous phase and, as dispersed phase, a water-in-oil emulsion in the form of droplets (G1), each droplet (G1) comprising a continuous fatty phase and at least one, preferably a single droplet (G2) comprising an internal aqueous phase, said droplets (G1) and (G2) being such as defined above, to encapsulate at least one hydrophilic compound, in particular a hydrophilic active cosmetic substance and optionally at least one lipophilic compound, in particular a lipophilic active cosmetic substance.

The expressions «between . . . and . . . », «from . . . to . . . » and «ranging from . . . to . . . » are to be construed limits included, unless otherwise specified.

The quantities of ingredients given in the examples are expressed in weight percentage relative to the total weight of the emulsion, unless otherwise indicated.

The following examples illustrate the present invention but do not limit the scope thereof.

### EXAMPLES

### Example 1: Double Emulsion of the Invention with a Heat-Sensitive Gelling Agent in the Fatty Phase of Droplets (G1)

Below, composition of the external aqueous phase (OF), fatty phase (MF) and internal aqueous phase (IF):

| Phase | Trade name | Supplier | INCI name | % w/w Phases |
|---|---|---|---|---|
| OF | EXTERNAL AQUEOUS GEL PHASE | | sub-total | 100.00 |
| | Osmosed water | / | Aqua | Q.S.* |
| | Microcare PE | THOR | Phenoxyethanol | 0.93 |
| | Microcare PTG | THOR | Pentylenglycol | 2.33 |
| | Glycerine codex | INTERCHIMIE | Glycerin | 17.49 |
| | (99%) | | | |
| | EDETA BD | BASF | Disodium EDTA | 0.04 |
| | Tego carbomer 340 | EVONIK | Carbomer | 0.09 |
| | FD | | | |
| | Carbopol Ultrez 21 | LUBRIZOL | Acrylates/C10-30 Alkyl Acrylate | 0.20 |
| | Polymer | | Crosspolymer | |
| | Zemea propanediol | DUPONT TALE ET | Propanediol | 5.83 |
| | | LYLE | | |
| | Butylene glycol | INTERCHIMIE | Butylen glycol | 5.83 |
| | Sodium Hydroxide | PANREAC | Sodium Hydroxyde | 0.01 |
| | Pellets PRS codex | | | |
| MF | OILY PHASE | | sub-total | 100.00 |
| | DUB ININ Grade A | STERAINERIE | Isononyl Isononanoate | 38.31 |
| | | DUBOIS | | |
| | Rheopearl KL2 | MYOSHI EUROPE | Dextrin palmitate | 20.00 |
| | DUB DPHCC | STEARINERIE | Dipentaerythrityl hexacaprylate/ | 38.27 |
| | | DUBOIS | hexacaprate | |
| | Fragrance | | Fragrance | 3.24 |
| | CAS-3131 | NUSIL | Amodimethicone | 0.18 |
| IF | INTERNAL AQUEOUS GEL PHASE | | sub-total | 100.00 |
| | Osmosed water | / | Aqua | Q.S. |
| | Microcare PE | THOR | Phenoxyethanol | 0.93 |
| | Microcare PTG | THOR | Pentylenglycol | 2.33 |
| | Glycerine codex | INTERCHIMIE | Glycerine | 17.49 |
| | (99%) | | | |
| | EDETA BD | BASF | Disodium EDTA | 0.04 |
| | Carbopol Ultrez 21 | LUBRIZOL | Acrylates/C10-30 Alkyl Acrylate | 0.20 |
| | Polymer | | Crosspolymer | |
| | Zemea propanediol | DUPONT TATE ET | Propanediol | 5.83 |
| | | LYLE | | |
| | Butylene glycol | INTERCHIMIE | Butylene glycol | 5.83 |
| | Sodium Hydroxide | PANREAC | Sodium Hydroxide | 0.01 |
| | Pellets PRS codex | | | |
| | Unicert Blue 05601-J | Sensient | CI 42090 | 0.00078 |

*Quantum Satis - sufficient quantity.

Below, composition of the sodium hydroxide solution (BF):

| | Trade name | Supplier | INCI name | % w/w |
|---|---|---|---|---|
| BF | Osmosed water | | | Q.S. |
| | Sodium Hydroxide | PANREAC | Sodium Hydroxide | 0.42 |
| | Pellets PRS codex | | | |
| | Total | | | 100.00 |

The preparation of each of the above phases lies within the general reach of skilled persons.

The proportions of the different phases of the final emulsion (PF) are given in the table below.

| | % phases in PF |
|---|---|
| IF | 9.25% |
| MF | 9.25% |
| OF | 76.51% |
| BF | 4.99% |

Experimental Device:

The equipment required to produce the emulsion in Example 1 is composed of: 4 syringe pumps (one for OF, MF, IF and BF), a syringe heater (for MF), a thermostatic bath, a concentric design 3-way microfluidic device (or nozzle) having coaxial outlets with, from the innermost channel to the outermost channel:
the first channel is dedicated to IF throughput;
the second channel is dedicated to MF throughput; and
the third channel is dedicated to OF throughput.

The nozzle and the line conveying the oily phase (MF) are placed in a thermostatic bath heated to 85° C.

The microfluidic device is also adapted to add a sodium hydroxide solution (BF) after formation of droplets (G1) and (G2) for enhancement of OF viscosity.

The flow rates, from the outermost channel to the innermost channel of the nozzle, under consideration for the different phases are the following:
OF: 76.70 mL/hr,
MF: 10.190 mL/hr,
IF: 9.273 mL/hr, et
BF: 5.000 mL/hr.

An emulsion according to Example 1 is of particular interest first in that it ensures particularly satisfactory encapsulation of the hydrophilic compounds by means of droplets (G2) but also of lipophilic compounds by means of droplets (G1). Secondly, the droplets (G1) and (G2) are macroscopic and monodisperse, and each droplet (G1) comprises a single droplet (G2). The emulsion is therefore given unique visual appearance. In addition, the emulsion is provided with a texture that is unique in the field of double emulsions which, for obvious reasons, meets continuing demand by consumers in this respect.

Also, the emulsion in Example 1 is of particular interest with regard to kinetic stability. Droplets (G1) additionally have satisfactory mechanical strength. An emulsion according to Example 1 is therefore able to remain stable over a timescale longer than 3 months, even longer than 6 months at 25° C. despite the absence of a shell for droplets (G2).

Optionally, the IF described above may also comprise a cationic polymer (PA2), in particular Carbomer Tego 340FD d′EVONIK in a content of 0.10 weight % relative to the total weight of IF. The presence of this cationic polymer (PA2) in IF is advantageous in that it allows reinforcing of the kinetic stability of the emulsion of the invention, in particular the mechanical strength of droplets (G2).

Alternatively, Rheopearl KL2 (INCI: Dextrin palmitate) can be replaced by Rheopearl MKL2 (INCI: Dextrin Myristate). The resulting oily phase (MF), and hence the end emulsion obtained, has the advantage of displaying improved transparency.

Also, the oily phase (MF) may additionally comprise at least one phenyl silicone oil (e.g. phenyltrimethicone, and better still a diphenylsiloxyphenyltrimethicone (INCI: Diphenylsiloxy Phenyl Trimethicone)) which further improves the transparency of droplets (G1) comprising a gelling agent of Rheopearl KL2 and/or Rheopearl MKL2 type, and therefore improves the transparency of the final composition.

### Example 2: Double Emulsion of the Invention with a Thixotropic Gelling Agent in the Fatty Phase of Droplets (G1)

Below, composition of the external aqueous phase (OF), fatty phase (MF) and internal aqueous phase (IF):

| | | | | % w/w |
|---|---|---|---|---|
| Phase | Trade name | Supplier | INCI name | Phases |
| OF | EXTERNAL AQUEOUS GEL PHASE | | sub-total | 100.00 |
| | Osmosed water | / | Aqua | Q.S. |
| | Microcare PE | THOR | Phenoxyethanol | 0.80 |
| | Microcare PTG | THOR | Pentylenglycol | 2.00 |
| | Glycerine codex (99%) | INTERCHIMIE | Glycerin | 20.00 |
| | EDETA BD | BASF | Disodium EDTA | 0.04 |
| | Tego carbomer 340 FD | EVONIK | Carbomer | 0.25 |
| | Sodium Hydroxide | / | Sodium Hydroxide | 0.11 |
| | Pellets PRS codex | | | |
| MF | OILY PHASE | | sub-total | 100.00 |
| | Myritol 318 | Ami Chimie | CAPRYLIC CAPRIC | 96.80 |
| | | | TRIGYLCERIDE | |
| | Aerosil R974 | Azelis | SILICA DIMETHYL | 3.00 |
| | | | SILYLATE | |
| | CAS-3131 | NUSIL | Amodimethicone | 0.20 |
| IF | INTERNAL AQUEOUS GEL PHASE | | sub-total | 100.00 |
| | Osmosed water | / | Aqua | Q.S. |
| | Microcare PE | THOR | Phenoxyethanol | 0.80 |
| | Microcare emollient PTG | THOR | Pentylenglycol | 2.00 |
| | EDETA BD | BASF | Glycerin | 0.04 |
| | Glycerine Codex 99% | Interchimie | Disodium EDTA | 20.00 |
| | 10% sodium hydrox. sol. | / | Acrylates/C10-30 Alkyl | 0.11 |
| | | | Acrylate Crosspolymer | |
| | Unicert Blue 05601-J | Sensient | CI 42090 | 0.45 |
| | 0.1% | | | |

The preparation of each of the above phases lies within the general reach of skilled persons.

The proportions of the different phases of the final emulsion (PF) are given in the table below.

| | % phases in PF |
|---|---|
| IF | 3.57% |
| MF | 10.71% |
| OF | 85.71% |

Experimental Device:

The equipment required to produce the emulsion in Example 2 is the same as used for Example 1, with the exception that Example 2 does not have recourse to the use of a syringe heater or thermostatic bath.

The flow rates under consideration for the different phases are the following:
OF: 120 mL/hr,
MF: 15 mL/hr, and
IF: 5 mL/hr.

An emulsion according to Example 2 is of particular interest first in that it ensures satisfactory encapsulation of hydrophilic compounds by means of droplets (G2) but also of lipophilic compounds by means of droplets (G1), and secondly it is provided with unique visual appearance and texture in the field of double emulsions which, for obvious reasons, meets growing demand by consumers in this respect.

In addition, an emulsion according to Example 2 is of particular interest with regard to kinetic stability since droplets (G1) have sufficient mechanical strength to remain intact over a timescale longer than 3 months, even longer than 6 months at 25° C.

Optionally, the IF described above may further comprise a cationic polymer (PA2), in particular Carbomer Tego 340FDby 'EVONIK in a content of 0.10 weight % relative to the weight of the IF. The presence of this cationic polymer (PA2) in IF is advantageous in that it allows further reinforcing of the kinetic stability of the emulsion of the invention, in particular the mechanical strength of droplets (G2).

1. A water-in-oil-in-water emulsion comprising an external continuous aqueous phase and a water-in-oil emulsion in the form of droplets, each droplet comprising a continuous fatty phase and at least one droplet comprising an internal aqueous phase, said continuous fatty phase containing at least one gelling agent,
said droplets comprising a shell formed of at least one anionic polymer and at least one cationic polymer, and optionally
said droplets comprising a shell formed of at least one anionic polymer, the same as or differing from, and of at least one cationic polymer.

2. The emulsion according to claim 1, wherein the gelling agent is selected from among organic or mineral, polymeric or molecular lipophilic gelling agents; fats solid at ambient temperature and pressure; and mixtures thereof.

3. The emulsion according to claim 1, wherein the gelling agent is selected from the group consisting of: polyacrylates; esters of sugar/polysaccharide and fatty acid(s), esters of inulin and fatty acid(s), esters of glycerol and fatty acid(s); polyamides; and mixtures thereof.

4. The emulsion according to claim 3, wherein the gelling agent is selected from the group consisting of: dextrin palmitates, dextrin myristates, dextrin palmitates/ethylhexanoates, and mixtures thereof.

5. The emulsion according to claim 1, wherein the gelling agent is selected from the group consisting of: optionally modified clays, silicas, and mixtures thereof.

6. The emulsion according to claim 1, comprising from 0.5% to 99.99% by weight of gelling agent(s) relative to the total weight of the fatty phase of droplets.

7. The emulsion according to claim 1, wherein the fatty phase of droplets comprises at least one oil selected from the group consisting of: hydrocarbon oils of vegetable origin, hydrocarbon oils of animal origin, synthetic esters and ethers, straight-chain or branched hydrocarbons of mineral or synthetic origin, silicone oils, fatty alcohols having 8 to 26 carbon atoms, partly hydrocarbon and/or silicone fluorinated oils and mixtures thereof.

8. The emulsion according to claim 1, comprising from 0% to 99.49% by weight of oil(s) relative to the total weight of the fatty phase of droplets.

9. The emulsion according to claim 1, wherein the anionic polymer(s), and optionally the anionic polymer(s), are polymers comprising monomeric units having at least one carboxylic acid function.

10. The emulsion according to claim 1, comprising from 0.01% to 5% by weight of anionic polymer(s), and optionally of anionic polymer(s) relative to the total weight of said emulsion.

11. The emulsion according to claim 1, wherein the cationic polymer has the following formula (I): 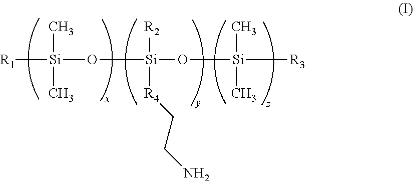
wherein:
R₁, R₂ and R₃ are each independently OH or CH₃;
R₄ is a —CH₂— group or —X—NH— group where X is a C₃ or C₄ divalent alkylene radical;
x is an integer between 10 and 5 000;
y is an integer between 2 and 1 000; and
z is an integer between 0 and 10.

12. The emulsion according to claim 1, comprising from 0.01% to 10% by weight of cationic polymer(s) relative to the total weight of the fatty phase of droplets.

13. The emulsion according to claim 1, wherein the volume fraction ρ (IF/(IF+MF)) is between 0.1 and 0.7, where:
IF is the total volume of droplets, and
MF is the total volume of droplets.

14. The emulsion according to claim 1, wherein the internal aqueous phase of droplets comprises at least one additional compound and/or active substance.

15. The emulsion according to claim 1, further comprising at least one active substance selected from among hydrating agents, healing agents, depigmenting agents, UV filters, peeling agents, antioxidants, active substances stimulating the synthesis of dermal and/or epidermal macromolecules, dermo-relaxants, anti-perspirants, soothing agents, anti-age agents, fragrances and mixtures thereof.

16. The emulsion according to claim 1, wherein the size of droplets is greater than 500 μm and/or the size of droplets is greater than 10 μm.

17. The emulsion according to claim 1, said emulsion not containing any surfactant.

18. A method for preparing an emulsion according to claim 1, comprising the following steps:
a) placing an aqueous fluid FE1 in contact with an oily fluid F1;
b) forming a water-in-oil emulsion composed of droplets, formed by aqueous fluid FE1, dispersed in a fatty phase formed by fluid F1; and
c) forming droplets, each droplet being composed of at least one droplet, by contacting the water-in-oil emulsion obtained at step b) with an aqueous fluid FE2;
wherein:
the aqueous fluid FE1 at least comprises water and optionally at least one anionic polymer;
the oily fluid F1 comprises at least one gelling agent and at least one cationic polymer, and optionally at least one oil; and
the aqueous fluid FE2 at least comprises water and at least one anionic polymer, the same as or differing from the anionic polymer.

19. The method according to claim 18, wherein at least the fluid F1, even aqueous fluid FE1, are heated to a temperature of 40° C. to 150° C. to perform step a), even steps b) and c), and optionally fluid FE2 is heated to a temperature of 40° C. to 150° C. to perform step c).

20. A composition comprising at least one emulsion according to claim 1 and a physiologically acceptable medium.

21. A non-therapeutic method for the cosmetic treatment of keratinous material comprising at least one step to apply onto said keratinous material at least one emulsion according to claim 1 or at least one composition according to claim 20.

22. (canceled)

23. A method for encapsulating at least one hydrophilic compound, and optionally at least one lipophilic compound, comprising contacting said hydrophilic compound with a water-in-oil-in-water emulsion, comprising an external continuous aqueous phase and a water-in-oil emulsion in the form of droplets, each droplet comprising a continuous fatty phase and at least one droplet comprising an internal aqueous phase, said droplets and being such as defined in claim 1.
